(19) United States
(12) Patent Application Publication
(10) Pub. No.: US 2017/0035733 A1
(43) Pub. Date: Feb. 9, 2017

(54) PHARMACEUTICAL COMPOSITION COMPRISING LACOSAMIDE AND LEVETIRACETAM

(71) Applicant: UCB PHARMA GMBH, Monheim (DE)

(72) Inventors: Benjamin THOORENS, Brussels (BE); Andrew FADDEN, Slough (GB); René Pierre PINARD, Brussels (BE); Florent ROBIN, Brain-l'Alleud (BE); Martin Alexander SCHUBERT, Brussels (BE); Frank TENNIGKEIT, Monheim (DE); Serge CUYPERS, Brain-I'Alleud (BE)

(73) Assignee: UCB PHARMA GMBH, Monheim (DE)

(21) Appl. No.: 15/039,313

(22) PCT Filed: Nov. 28, 2014
(86) PCT No.: PCT/EP2014/075904

§ 371 Date: May 25, 2016

(30) Foreign Application Priority Data
Nov. 29, 2013 (EP) 13005561.9
Jan. 2, 2014 (EP) 14000007.6

(51) Int. Cl. A61K 31/4015; A61K 9/00; A61K 9/20; A61K 31/165

(52) U.S. Cl. CPC A61K 31/4015; A61K 31/165; A61K 9/0053; A61K 9/0019; A61K 9/20; A61K 9/2009; A61K 9/2027; A61K 9/2095

(57) ABSTRACT

The present application relates to a fixed dose combination comprising lacosamide and levetiracetam, as well as to dosage regimens including such fixed dose combinations. The fixed dose combinations are suitable for the oral or parenteral treatment of various diseases, including in particular epilepsy and/or epileptic seizures.

### BACKGROUND OF INVENTION

Lacosamide (LCM, R-2-Acetamido-N-benzyl-3-methoxypropionamide) is an anticonvulsive drug which is approved for adjunctive therapy of partial onset seizures (POS), with and without secondary generalization, in many countries of the world, including the US and EU. Moreover, lacosamide was approved recently for the monotherapy of POS in the US. Lacosamide is available as immediate release (“IR”) tablet (50, 100, 150 and 200 mg) for twice daily administration, as oral solution and as i.v. solution, and is commercialized under the tradename Vimpat®.

LCM is effective in a significant amount of epilepsy patients, who are refractory to or insufficiently controlled by other AEDs (Ben-Menachem, et al, Epilepsia, 2007, 48, 1308-1317.). LCM was found to be particularly effective, if it was given to insufficiently controlled epilepsy patients who were only on one or two previously administered AEDs (Villanova et al, Epilepsy & Behaviour, 29, 2013, 349-356).

The risk for pharmacokinetic or pharmacodynamics drug-drug interaction is low for lacosamide, which has been shown to have no significant effect on plasma levels of other AEDs such as e.g., carbamazepine, levetiracetam, lamotrigine, topiramate, valproate, zonisamide, gabapentin, and phenytoin (Doty et al, Ann Ny Acad Sci, 1291, 2013, 56-68; Patsalos, Clin Pharmacokinet, EPub Jun. 20, 2013).

Hence, in order to make use of these favorable properties of LCM and in order to reduce the pill burden of epilepsy patients who are in need of an anticonvulsive combination therapy, a fixed dose dual combination therapy comprising LCM would be helpful. The aim of the present disclosure was thus to provide an effective and safe pharmaceutical composition being a dual (combined) fixed-dose combination (“FDC”) comprising LCM, such as, for example, an oral FDC (“oFDC”).

WO 2007-144195 discloses that LCM shows beneficial efficacious effects in combination with various AEDs in an animal model of epilepsy. Dual combinations of three different doses of LCM were tested in the 6 Hz epilepsy model with different ED50-adapted doses of each of lamotrigine, carbamazepine, levetiracetam, brivaracetam, topiramate, gabapentin, phenytoin and valproic acid. Based on the isobolographical analyses, lamotrigine, carbamazepine, levetiracetam, brivaracetam, topiramate, and gabapentin were considered to be good combination partners from the efficacy and safety perspective. Synergistic effects of all three dose combinations tested were seen for dual combinations of LCM with levetiracetam (LEV, (2R)-2-(2-oxopyrrolidin-1-yl)butanamide), and with carbamazepine but synergistic effects were also identified for two of three dose combinations of LCM with lamotrigine, topiramate and gabapentin. However, the dosages envisaged for these combinations are high in most cases, and were likely to lead to oFDCs with a significant size, making them inconvenient for the patients to swallow.

Doses proposed in WO 2007-144195 for LEV in such combinations with LCM were in the range of 1-3 g/day, doses for gabapentin were even higher (900-3600 g/day), and doses of carbamazepine were 400-1600 g, which was still expected to make the development of a once or twice daily oFDC with a reasonable size very difficult.

According to a retrospective study of clinical data reported by Villanueva et al (supra), the highest seizure-free rates were achieved when LCM was given to patients who were on valproate, LEV and lamotrigine, while the combination of LCM with LEV was associated with significant fewer withdrawals from treatment because of adverse events than any other combination of LCM.

LEV is presently available as tablet in dosages of 250 mg, 500 mg, 750 mg and 1000 mg to be administered twice daily (commercialized, inter alia under the tradename Keppra®), as i.v. solution, as oral solution, and in the U.S. as extended release tablets of 500 mg and 750 mg, which are to be administered once daily.

The daily amount of LEV usually needed is the gram range (1-3 g/day). An oFDC of additive or more than additive size compared to the presently commercialized tablets of lacosamide and levetiracetam would thus result in an oFDC size which is inconvenient for the patient to swallow. Instead, the size of a potential oFDC comprising both drugs should not exceed, but preferably be lower than the added volume of the commercially available Keppra® and Vimpat® tablets.

However, a person skilled in the art could have expected that minimizing the oFDC size by significantly reducing the total amount of excipients, compared to e.g. the commercial lacosamide tablets, could lead to physicochemical LEV-LCM drug-drug interactions. Moreover, certain amounts of excipients are usually needed to control the stability of the formulation and the robustness of the drug release profiles. For example, the amount of excipients in the presently available Vimpat® tablets is well above 50 wt % (see Comparative Example 1).

Furthermore, in order to provide an oFDC which can substitute the patient's preexisting LEV and LCM medication, the oFDC should preferably be bioequivalent to the commercial tablet formulations of both, LCM and LEV. Given the complexity of such a combined dual drug formulation, it seemed thus very uncertain or even unlikely that the amount of excipients in the oFDC could be sufficiently reduced while simultaneously meeting the requirements of the above specified target profile of a potential LEV+LCM oFDC.

### DESCRIPTION OF THE INVENTION

It has been found, surprisingly, that LCM and LEV do not adversely physicochemically interact with each other even in the absence of significant amounts of excipients.

Accordingly, the present invention provides pharmaceutical compositions (FDCs) suitable for the joint administration of LCM and LEV, said FDC comprising (a) LCM in an amount of 50 mg-400 mg, and preferably in an amount selected from 50 mg, 100 mg, 150 mg and 200 mg and, (b) LEV in an amount of 250-1500 mg, preferably selected from among 250 mg, 500 mg, 750 mg and 1000 mg.

The FDCs of the present invention can be any LEV-LCM FDC, and are preferably an injectable or infusible solution (“iFDC”), or particularly preferably an oral FDC (“oFDC”). In one aspect, the FDC of the present invention is a solid oFDC.

It has been surprisingly found by the inventors of the present application that the amount of excipients can be limited to a low amount such as e.g. below 15 wt % or below 10 wt % or even below about 5 wt % of the total weight of the formulation, thus allowing for a solid oFDC with a convenient size. Despite the low content of excipients, these FDCs, surprisingly provide robust release profiles, do not show interactions between LCM and LEV, and provide excellent physical stability, such as e.g. in the case of the FDC being a tablet, it shows very suitable hardness and friability properties (Tables 8 a and b).

In an alternative embodiment of present invention, the FDC is an injectable or infusible FDC (collectively “iFDC”).

Oral Fixed Dose Combinations (oFDC)

In a preferred embodiment of the present invention, the FDC of the present invention is an oFDC for the combined administration of LCM and LEV. Preferably, the oFDC is a solid oFDC, such as a multiple unit dosage form or a single unit dosage form, and particularly preferable a tablet.

The term “solid oFDC” refers to an oFDC in which the amount of solvent/water is below 10 wt %, preferably below 5 wt %, more preferably below 3 wt %, and even more preferably below 1 wt %.

“Multiple unit dosage forms (“MUDs”) comprise powders/particles, pellets, minitablets, sprinkles or granules, which may be covered with coatings prior to further processing and/or administration, and/or which may be packed into sachets or capsules. “Multiple unit dosage forms” may also be compressed to dispersible tablets consisting of powders/particles, pellets, minitablets, or granulates, or may be “sprinkled” on soft food or drinks, and swallowed without chewing (“sprinkle delivery”), including tube feeding. Each entity of the “multiple dosing units” (e.g. each pellet, granulate or mini-tablet) is preferably a full functional unit showing in average the in-vitro dissolution properties further defined in this specification. Typically, multiple unit dosage forms have sizes below 5 mm, preferably below 4 mm, and more preferably below 3 mm, and a drug load of 50 mg or less, 25 mg or less, preferably 15 mg or less, and even more preferred 10 mg active ingredient or less. Accordingly, a multitude of e.g. pellets or mini-tablets needs to be administered in order to provide the effective dose to be administered at a given time. For example, in order to administer 500 mg of a given drug combination 50 mintablets with a drug load of 10 mg may need to be administered. “Minitablets” comprise flat or slightly curved tablets with a diameter of about 1-4 mm, preferably with a diameter of 2-3 mm. Minitablets may be pressed into tablets or placed into sacchets or capsules; they may be produced via tableting. “Pellets” comprise free flowing spherical or semispherical bead-like solid units with a particle size of between about 0.5 and 1.5 mm, preferably between about 0.6 and 1 mm. Pellets may be produced via fluid bed granulation, extrusion spheronization, or spray drying technologies. “Granules” comprise irregular sized small particles of sizes typically below 1 mm, or even below 0.5 mm, which are usually produced via granulation. If used for “sprinkle delivery”, the MUDs have a maximum bead size of about 2.5 mm, preferably of about 2 mm, more preferably of about 1.5 mm.

“Single unit dosage forms” are physical entities individually showing the dissolution properties disclosed herein. Upon disintegration single unit dosage forms such as e.g. tablets or dragees, usually do not disperse into separate functional units. Typically, one single unit dosage form contains at least 33%, more preferably at least 50%, and even more preferably 100% of the drug to be administered to a patient at a given time, i.e. the drug load of a single unit dosage form is usually between 50 mg and 1500 g, depending on the drug to be administered.

In one aspect, the FDCs described herein may comprise isotopic analogs and/or polymorphs of LCM and/or LEV. The term “isotopic analogs” includes all suitable isotopic variations of LCM and LEV wherein at least one atom of LCM and/or LEV, respectively, is replaced by an atom having the same atomic number but an atomic mass different from the atomic mass usually found in nature, with the most abundant isotope(s) being preferred. Examples of isotopes that can be incorporated into LCM or LEV include isotopes of hydrogen, carbon, nitrogen, and oxygen such as e.g. H², H³, C¹³, N¹⁵, O¹⁸, respectively, with deuterium (H²) being preferred. Isotopic analogs of LCM, e.g. deuterated LCM or LEV, can be prepared e.g. by conventional procedures using appropriate isotopic variations of suitable reagents. The term “polymorphs” refers to distinct crystal forms of the drugs. Disclosure of suitable polymorphs of LCM can be found in e.g. WO 2012/072256.

It has been surprisingly found by the inventors of the present application that LCM and LEV do not adversely interact with each other even in high dosages and in the absence of larger amounts of excipients. It was also found, unexpectedly, that a robust release profile of LEV and LCM, and a physically stable formulation can be achieved despite a total drug load of as much as more than 75 wt %, more than 80 wt %, preferably more than 85 wt %, and particularly preferably more than 90 wt %, more than 93 wt %, or more than 95 wt %. In one aspect, the drug load is between 90 wt % and 97 wt %, preferably between 93 wt % and 96 wt %. The term “drug load” as used herein refers to the total amount of drug (LEV+LCM) compared to the total weight of the oFDC.

Surprisingly, it has been found that in an oFDC, in particular in a solid oFDC, a certain quantity of LEV works as super-disintegrant which promotes the release of LCM, such that LEV can substitute for a certain amount of other disintegrants in said oFDC comprising both drugs. This is the case, for example, if LEV is present in the solid oFDC in an amount (wt/wt) at least equal to LCM, see Example 15. Preferably, the amount of LEV is at least twice the amount of LCM (wt/wt). It is also preferred that the amount of LEV is less than twenty the amount of LCM (wt/wt). Further, it has been found, unexpectedly, that LEV in combination with a suitable lubricant, improves the hardness of the solid oFDC, in particular of a fixed dose tablet, such that the use of a binder can be omitted in the solid oFDC.

As a consequence of these surprising findings, the amount of excipients in the solid oFDC could be reduced even further, thus leading to drug loads of more than 90 wt %, more than 91 wt %, more than 92 wt %, more than 93 wt %, more than 94 wt % or even more than 95 wt % of the solid oFDC, while still maintaining the target product profile described further above. In one aspect, the drug load in the solid oFDC is between 90 wt % and 97 wt %, preferably between 93 wt % and 96 wt %.

Accordingly, one aspect of the present invention relates to a pharmaceutical composition for the combined oral administration of LCM+LEV, said composition comprising at least about 80 wt %, preferably more than about 85 wt %, particularly preferably more than 90 wt % (e.g. between 90 and 97 wt %), even more preferably at least about 93 wt % (e.g. between 93 and 96 wt %), even more preferably at least about 95 wt % of active ingredient, which active ingredient consists of a combination of LEV and LCM in a ratio (wt/wt) of about 1:1 to about 20:1, preferably in a ratio of LEV:LCM of 2:1 to about 20:1 and more preferably in a LEV:LCM ratio of 2.5:1 to 15:1, of 3:1 to 15:1 or of 5:1 to 15:1, such as e.g. 2:1, 2.5:1, 3:1, 4:1, 5:1, 6:1, 7:1, 8:1, 9:1, 10:1, 11:1, 12:1 13:1, 14:1 or 15:1.

In this application, the term “combined administration” shall mean that LEV and LCM are being released from the joint composition (e.g. from the oFDC) in a coordinated fashion, either sequentially, or more or less simultaneously. For example, in one embodiment the LEV release may start somewhat earlier than the LCM release, or vice versa, but the release of the other active ingredient may start shortly thereafter, thus leading to an overlapping release profile of both compounds from the oFDC. In one embodiment, the release of both compounds may take place sequentially, i.e. first LCM may be released and once finalized, LEV may be released subsequently, or vice versa, as further described herein. In a preferred embodiment, LCM and LCM will be released from the oFDC in an essentially simultaneous immediate release fashion.

In a preferred example at least 85% of both drugs may be released from the oFDC in 15 minutes (immediate release, “IR”). It is also preferred that at least 97%, or at least 98% or even about 100% of both drugs are dissolved within 30 minutes, or even already in about 20, or already in 15 minutes if measured in vitro using standard dissolution assays as further described herein (see Example 15). Such immediate release dissolution profiles may lead to average maximum plasma (Cmax) levels of LCM after about 1-3 hours (hrs), preferably after 1-2 hrs, and of LEV after about 1-3 hrs, preferably after about 1-2 hrs following a single administration of the IR oFDC to a patient.

Further release profiles of the combined administration are described herein and are also encompassed by the scope of the invention.

The pharmaceutical compositions of the present invention may be given once daily or twice daily, depending on the release profile of LCM and LEV. In a preferred embodiment, the oFDC provides the immediate release of LCM and LEV, and is to be administered twice daily in order to achieve an effective dose of LEV and LCM over the full treatment period, i.e. for about 24 hours.

Suitable dosages of LCM in the pharmaceutical composition (oFDC), in particular of the IR oFDCs may be between 50 and 400 mg, and preferably be selected from 50 mg, 100 mg, 150 mg and 200 mg, whereas preferred LEV dosages may be between 250 mg and 1500 mg and be preferably selected from the group of 250 mg, 500 mg, 750 mg, and 1000 mg. Preferred exemplary dosage combinations of LEV+LCM are shown in Table 1:

| oFDC | Example 1 | Example 2 | Example 3 | Example 4 | Example 5 |
|---|---|---|---|---|---|
| LEV | 500 mg | 500 mg | 750 mg | 750 mg | 1000 mg |
| +LCM | 50 mg | 100 mg | 50 mg | 100 mg | 200 mg |

In one particular embodiment, dosages of LCM in the oFDC are 50 or 100 mg. In one embodiment, preferred dosages of LEV in the oFDC are 500 or 750 mg. In another embodiment preferred dosages of LCM are 50 mg, 100 mg, 150 mg or 200 mg, and preferred dosages of LEV are 500 mg, 750 mg. or 1000 mg. In one embodiment, dosages of LEV+LCM in the oFDC are preferably 50 LCM+250 mg LEV, 50 mg LCM+500 mg LEV; 50 mg LCM+750 mg LEV; 100 mg LCM+500 mg LEV; 100 mg LCM+750 mg LEV, or 200 mg LCM+1000 mg LEV. In one aspect, dosages of LEV+LCM in the oFDC are 150 mg LCM+500 mg LEV, 150 mg LCM+1000 mg LEV, or 200 mg LCM+500 mg LEV.

These oFDCs lead to convenient dosage regimens where patient's “pill burden”, i.e. the amount of oFDC units that patients need to take can be reduced to up to 50%. Suitable exemplary oFDC-saving dosage regimens and the resulting pill burden achieved are given in Table 2 below. The oFDC dosing regimens disclosed in column 3 of table 2 are preferred embodiments of the present invention.

TABLE 2
Examples of reduction in pill-burden through the use of the LCM/LEV FDC

| | LCM + LEV; strengths and | | |
|---|---|---|---|
| | number of commercially | LCM/LEV FDC, | Reduction in |
| | available Vimpat ® and | corresponding strengths | number of |
| LCM + LEV Daily | Keppra ® tablets (twice daily | and number of oFDCs | medication |
| dose | administration) | (twice daily administration) | units |
| 100 mg + 500 mg | 2 × [1 × 50 mg + 1 × 250 mg] | 2 × 50/250 mg | 50% |
| 100 mg + 1000 mg | 2 × [1 × 50 mg + 1 × 500 mg] | 2 × 50/500 mg | 50% |
| 100 mg + 1500 mg | 2 × [1 × 50 mg + 1 × 750 mg] | 2 × 50/750 mg | 50% |
| 200 mg + 500 mg | 2 × 1 × 100 mg + 1 × 250 mg] | 2 × 100/250 mg | 50% |
| 200 mg + 1000 mg* | 2 × [1 × 100 mg + 1 × 500 mg] | 2 × 100/500 mg | 50% |
| 200 mg + 1500 mg | 2 × [1 × 100 mg + 1 × 750 mg] | 2 × 100/750 mg | 50% |
| 200 mg + 2000 mg* | 2 × [1 × 100 mg + 1 × 1000 mg] | 2 × 100/1000 mg | 50% |
| | or | or | or |
| | 2 × [1 × 100 mg + 2 × 500 mg]** | 2 × [2 × 50 mg/500 mg]** | 33% |
| 200 mg + 3000 mg* | 2 × [1 × 100 mg + 2 × 750 mg] | 2 × [2 × 50/750 mg] | 33% |
| 300 mg + 1000 mg | 2 × [1 × 150 mg + 1 × 500 mg] | 2 × 150/500 mg | 50% |
| 300 mg + 1500 mg | 2 × [1 × 150 mg + 1 × 750 mg] | 2 × 150/750 mg | 50% |
| 300 mg + 2000 mg | 2 × [1 × 150 mg + 1 × 1000 mg] | 2 × 150/1000 mg | 50% |
| 300 mg + 3000 mg* | 2 × [1 × 150 mg + 2 × 750 mg] | 2 × 100 mg/750 mg + | 33% |
| | | 2 × 50 mg/750 mg | |
| | | or | |
| | | 2 × [2 × 75 mg/750 mg] | |
| 400 mg + 1000 mg | 2 × [1 × 200 mg + 1 × 500 mg] | 2 × 200/500 mg | 50% |
| 400 mg + 1500 mg | 2 × [1 × 200 mg + 1 × 750 mg] | 2 × 200/750 mg | 50% |
| 400 mg + 2000 mg* | 2 × [1 × 200 mg + 1 × 1000 mg] | 2 × 200/1000 mg | 50% |
| | or | or | or |
| | 2 × [1 × 200 mg + 2 × 500 mg]** | 2 × [2 × 100 mg/500 mg]** | 33%** |
| 400 mg + 3000 mg* | 2 × [1 × 200 mg + 2 × 750 mg] | 2 × [2 × 100/750 mg] | 33% |

*combinations with an asterisk indicate the six most frequently prescribed combinations;
**alternative dosage regimes are indicated which may be convenient to patients who are no able or reluctant to swallow tablets with a drug load of 1 g or more. In these cases, two oFDCs have to be taken per administration.

Accordingly, one aspect of the present invention relates to a dosage regimen using the oFDCs of the present invention, wherein the number of medications compared to the treatment of single drug units of LEV and LCM is reduced by at least 25%, by at least 33%, and preferably by at least 50%.

One aspect of the present invention is an improved dosing regimen comprising an oral fixed dose combination of levetiracetam and lacosamide, for use as a medicine, preferably for use in the prophylaxis, alleviation and/or treatment of epileptogenesis, of an epileptic disorder and/or of epileptic seizures, most preferably in the therapy of partial onset seizures with and without secondary generalization, or in the treatment of primary generalized tonic clonic seizures, which use comprises the twice daily administration of one entity of said fixed dosage combination (such as e.g. one tablet or capsule), wherein said one entity provides the combined release of lacosamide and levetiracetam in dosages selected from (a) 50 mg LCM+250 mg LEV, (b) 50 mg LCM+500 mg LEV, (c) 50 mg LCM+750 mg LEV, (d) 100 mg LCM+500 mg LEV, (e) 100 mg LCM+750 mg LEV, and (f) 200 mg LCM+1000 mg LEV. Other suitable dosages for use in said dosage regimen are (g) 150 mg LCM+500 mg LEV, (h) 150 mg LCM+1000 mg LEV, (i) 200 mg LCM+500 mg LEV, (j) 100 mg LCM+250 mg LEV, (k) 100 mg LCM+1000 mg LEV, (l) 150 mg LCM+750 mg LEV, and (m) 200 mg LCM+750 mg LEV. As a consequence, in these particular dosing regimens only two oFDC entities need to be swallowed by the patient per day. Such treatment regimens reduce the pill burden compared to the twice daily immediate release LEV+LCM treatment regimen known in the art (i.e. Keppra® and Vimpat® IR tablets) by 50%. In a preferred embodiment, the oFDC entity is a single unit dosage form and particularly preferable a tablet, and even more preferable a tablet which releases LEV and LCM in immediate release fashion.

Notably all of these preferred dosage combinations in the present dosing regimen comprise levetiracetam-lacosamide wt/wt-ratios of 2.5:1 to 12.5:1, and thus fall into the range of fixed dose ratios described in WO 2007-144195 to show beneficial co-action in epilepsy models, when these preferred fixed dose ratios are calculated on the applicable ED50 values of levetiracetam and lacosamide, respectively. Hence, the inventive dosing regimen described herein do not only lead to a reduced pill burden for the patient compared to the state-of-the art treatment, but are also likely to show particularly beneficial pharmacological co-action.

Hence, one aspect of the present invention is an oral fixed dose combination (oFDC) comprising levetiracetam and lacosamide for use as a medicine, preferably for use in the prophylaxis, alleviation and/or treatment of epileptogenesis, of an epileptic disorder and/or of epileptic seizures, preferably in the therapy of partial onset seizures with and without secondary generalization, or in the treatment of primary generalized tonic clonic seizures, wherein the oral fixed dose combination is a tablet (“fixed dose tablet”), and wherein the prophylaxis, alleviation and/or treatment comprises the twice daily administration of one fixed dose tablet per administration, which fixed dose tablet provides the combined release of lacosamide and levetiracetam in a dosage selected from (a) 50 mg LCM+250 mg LEV, (b) 50 mg LCM+500 mg LEV, (c) 50 mg LCM+750 mg LEV, (d) 100 mg LCM+500 mg LEV, (e) 100 mg LCM+750 mg LEV, and (f) 200 mg LCM+1000 mg LEV, and from (g) 150 mg LCM+500 mg LEV, (h) 150 mg LCM+1000 mg LEV, and (i) 200 mg LCM+500 mg LEV, (j) 100 mg LCM+250 mg LEV, (k) 100 mg LCM+1000 mg LEV, (l) 150 mg LCM+750 mg LEV, and (m) 200 mg LCM+750 mg LEV.

In one embodiment, in order to reflect the six most commonly used dosage combinations of LCM and LEV, preferred dosage combinations of the oFDCs described herein are LCM/LEV of 50 mg LCM+500 mg LEV; 100 mg LCM+500 mg LEV; 50 mg LCM+750 mg LEV; 100 mg LCM+750 mg LEV; 100 mg LCM+1000 mg LEV and 200 mg LCM+1000 mg LEV.

In one aspect, the fixed dose tablet provides the release of both LCM and LEV in the particular dosages described hereinbefore in an immediate release fashion.

One aspect of the present invention is an improved dosing regimen comprising an oral fixed dose combination (oFDC) comprising levetiracetam and lacosamide for use as a medicine, preferably for use in the prophylaxis, alleviation and/or treatment of epileptogenesis, of an epileptic disorder and/or of epileptic seizures, most preferably in the treatment of partial onset seizures with and without secondary generalization, or in the treatment of primary generalized tonic clonic seizures, which comprises the twice daily administration of two entities per administration of the fixed dosage combination (such as e.g. tablets or capsules), each entity of which provides the combined release of lacosamide and levetiracetam in dosages selected from (a) 50 mg LCM+750 mg LEV, (b) 100 mg LCM+750 mg LEV, (c) 50 mg LCM+500 mg LEV, (d) 100 mg LCM+500 mg LEV, and (e) 75 ml LCM and 750 mg LEV. Such dosing regimens which include the uptake of 4 oFDCs per day reduces the pill burden compared to the dosing regimen available in the art (i.e. Keppra® and Vimpat® IR tablets) by at least 33% (see e.g. Table 2). In a preferred embodiment, the oFDCs are single unit dosage forms, in particular tablets, and even more preferred the oFDCs are tablets which release LEV and LCM in immediate release fashion.

Accordingly, one aspect of the present invention is an oral fixed dose combination comprising levetiracetam and lacosamide for use as a medicine, preferably for use in the prophylaxis, alleviation and/or treatment of epileptogenesis, an epileptic disorder and/or epileptic seizures, preferably in the treatment of partial onset seizures with and without secondary generalization, or in the treatment of primary generalized tonic clonic seizures, wherein the fixed dose combination is a fixed dose tablet, and wherein the prophylaxis, alleviation and/or treatment comprises the twice daily administration of two fixed dose tablets per administration, each of which provides the combined release of lacosamide and levetiracetam in a dosage selected from (a) 50 mg LCM+750 mg LEV, (b) 100 mg LCM+750 mg LEV, (c) 50 mg LCM+500 mg LEV, (d) 100 mg LCM+500 mg LEV and (e) 75 ml LCM and 750 mg LEV. In one aspect, the fixed dose tablet provides the release of both LCM and LEV in the particular dosages described hereinbefore in an immediate release fashion.

In a preferred embodiment the dosing regimens described herein comprise the administration of two or four oFDC entities per day, which oFDCs comprise identical dosages of LEV+LCM. For example, if a single oFDC entity administered in the morning contains, 100 mg LCM+500 mg LEV, the single oFDC entity to be taken in the evening preferably also contains 100 mg LCM+500 mg LEV. Likewise, if two oFDC entities administered in the morning each contain 100 mg LCM+750 mg LEV, then the two oFDC entities to be taken in the evening also preferably each contain 100 mg LCM+750 mg LEV.

In one preferred aspect the fixed dose tablets of the dosing regimens of the present invention have a drug load of more than 75 wt %, preferably more than 80 wt %, more than 85 wt %, particularly preferably more than 90 wt %, even more preferably more than 93 wt %, or even more than 95 wt %. Particularly preferably, the fixed dose tablets of the dosing regimens have a drug load of at least 90 wt %, at least 93 wt %, or even of 95 wt % or more. In one aspect, the drug load is between 90 wt % and 97 wt %, preferably between 93 wt % and 96 wt %.

One aspect of the present invention relates to a method of preventing, alleviating and/or treating of an epileptic disorder and/or of epileptic seizures, or of epileptogenesis, and to a method of treating partial onset seizures with and without secondary generalization, or to a method of treating primary generalized tonic clonic seizures, comprising administering to a patient in need thereof an oral fixed dose combination of levetiracetam and lacosamide, wherein said administration comprises the twice daily administration of one entity of said fixed dose combination (such as e.g. one tablet or capsule), wherein said one entity provides the combined release of lacosamide and levetiracetam in dosages selected from (a) 50 mg LCM+250 mg LEV, (b) 50 mg LCM+500 mg LEV, (c) 50 mg LCM+750 mg LEV, (d) 100 mg LCM+500 mg LEV, (e) 100 mg LCM+750 mg LEV, and (f) 200 mg LCM+1000 mg LEV. Other suitable dosages of said methods are (g) 150 mg LCM+500 mg LEV, (h) 150 mg LCM+1000 mg LEV, and (i) 200 mg LCM+500 mg LEV, (j) 100 mg LCM+250 mg LEV, (k) 100 mg LCM+1000 mg LEV, (l) 150 mg LCM+750 mg LEV, and (m) 200 mg LCM+750 mg LEV. In a preferred embodiment, the oFDC entity administered is a single unit dosage form and particularly preferable a tablet, and even more preferable a tablet which releases LEV and LCM in immediate release fashion.

One aspect of the present invention relates to a method of preventing, alleviating and/or treating of an epileptic disorder and/or of epileptic seizures, or of epileptogenesis, and to a method of treating partial onset seizures with and without secondary generalization, or to a method of treating primary generalized tonic clonic seizures, comprising administering to a patient in need thereof an oral fixed dose combination of levetiracetam and lacosamide, wherein said oral fixed dose combination is a tablet (“fixed dose tablet”), and wherein said administration comprises the twice daily administration of one fixed dose tablet per administration, which fixed dose tablet provides the combined release of lacosamide and levetiracetam in a dosage selected from (a) 50 mg LCM+250 mg LEV, (b) 50 mg LCM+500 mg LEV, (c) 50 mg LCM+750 mg LEV, (d) 100 mg LCM+500 mg LEV, (e) 100 mg LCM+750 mg LEV, and (f) 200 mg LCM+1000 mg LEV. Other suitable dosages in this method are (g) 150 mg LCM+500 mg LEV, (h) 150 mg LCM+1000 mg LEV, and (i) 200 mg LCM+500 mg LEV, (j) 100 mg LCM+250 mg LEV, (k) 100 mg LCM+1000 mg LEV, (l) 150 mg LCM+750 mg LEV, and (m) 200 mg LCM+750 mg LEV. In one aspect, the fixed dose tablet provides the release of both LCM and LEV in the particular dosages described hereinbefore in an immediate release fashion.

One aspect of the present invention relates to a method of preventing, alleviating and/or treating of an epileptic disorder and/or of epileptic seizures, or of epileptogenesis, and to a method of treating partial onset seizures with and without secondary generalization, or to a method of treating primary generalized tonic clonic seizures, comprising administering to a patient in need thereof an oral fixed dose combination of levetiracetam and lacosamide, wherein said administration comprises the twice daily administration of two fixed dose tablets per administration, each of which provides the combined release of lacosamide and levetiracetam in a dosage selected from (a) 50 mg LCM+750 mg LEV, (b) 100 mg LCM+750 mg LEV, (c) 50 mg LCM+500 mg LEV, (d) 100 mg LCM+500 mg LEV and (e) 75 ml LCM and 750 mg LEV. In one aspect, the fixed dose tablet provides the release of both LCM and LEV in the particular dosages described hereinbefore in an immediate release fashion.

In a preferred embodiment, the methods of treatment described herein comprise the administration of two or four identical oFDC entities per day. For example, if a single oFDC entity administered in the morning contains, by way of example, 100 mg LCM+500 mg LEV, the single oFDC entity to be taken in the evening also contains 100 mg LCM+500 mg LEV. Likewise, if two oFDC entities administered in the morning each contain 100 mg LCM+750 mg LEV, then the two oFDC entities to be taken in the evening also preferably each contain 100 mg LCM+750 mg LEV. Also, if two FDC entities are to be taken in the morning and two in the evening, preferably all four entities have the same drug content. This prevents that patients confuse potential different FDCs to be taken at a given time, which may lead to inaccurate dosing of the drugs. According to the dosage regimens described herein, substantially all dosage combinations achievable by combining the approved dosages of Vimpat® and Keppra® can also be achieved by the multiple administration of one and the same FDC with the appropriate drug content, see table 2 above. Alternatively, if exceptionally different FDC dosages are to be taken at a given time, such as e.g. one FDC comprising 100 mg LCM and 750 mg LEV and a second pill containing 50 mg LCM and 750 mg LEV, both twice a day, the different FDC doses may be marked by different colors or other indicators in order to prevent any mistake in patient's dosing.

In one aspect the oFDCs such as the fixed dose tablets of the methods of treatments of the present invention have a drug load of more than 75 wt %, more than 80 wt %, more than 85 wt %, more than 90 wt %, more than 93 wt %, or more than 95 wt %. Preferably, the fixed dose tablets of the dosing regimens have a drug load of at least 90 wt %, at least 93 wt %, or even of 95 wt % or more. In one aspect, the drug load is between 90 wt % and 97 wt %, preferably between 93 wt % and 96 wt %.

The solid oFDC according to the present invention may be a single unit dosage form, such as a tablet, or a multiple unit dosage form, such as minitablets, pellets or granules which may be packed into capsules.

Preferably the oFDC is single unit dosage form, and is particularly preferable a tablet.

Both active ingredients, LCM and LEV are preferably included in the same layer/matrix of the solid oFDC. In other words, the solid oFDC, preferably the tablet, comprises a monolayer/single matrix comprising both active ingredients as well as the excipients. As used herein, the terms “layer” and “matrix” are used interchangeably, unless expressly specified otherwise. Preferably both LCM and LEV are released from the same layer/matrix in immediate release rates as described hereinbefore.

In one embodiment, LCM and LEV may be delivered from a same matrix in a modified or delayed release course i.e. the release of both, LCM and LEV, is delayed compared to an IR formulation wherein typically substantially all of the compounds are released after 1 hours, or even after 15 minutes. In case of a modified release (MR) oFDC, bot LEV and LCM may preferably be released from the oFDC such that typically no more than about 50 wt %, preferably no more than 45 wt % of each of both compounds is released within one hour, between about 15 wt % and 60 wt % of each of the compounds is released after 2 hours, between about 30 wt % and 85 wt % is released within 4 hours, between about 55 and 100 wt % is released within 8 hours, and/or between 70 and 100 wt % is released within 12 hours, when measured in an in vitro dissolution assay as further specified herein. This may be achieved either by adding modified release polymers or other retarding agents to the matrix or by applying a release modifying coating to an immediate release matrix, or by a combination of release modifying components in the matrix and in the coating. For the purpose of this patent application, the terms “modified release” and “delayed release” are used interchangeably.

Such a modified or delayed release profile may advantageously lead to lower maximum plasma concentrations Cmax of the drugs, a reduction of the respective Cmax/Cmin ratio, an increased time Tmax to reach Cmax, and to potentially reduced side effects. LCM and LEV may be released from the matrix with delayed rates such that, for example, the maximum concentration of LCM in the patient would be reached at a time Tmax which is more than 2 or 3 hours, more than 4 hours, more than 5 hours, or even more than 6 hours after the administration of the oFDC to a patient, and/or such that the maximum concentration of LEV may be reached at a time Tmax which is more than 3, more than 4, more than 5, or after more than 6 hours after such administration.

In one preferred embodiment, LEV and LCM are comprised in the same layer/matrix of the oFDC, together with the excipients, and both active ingredients are released from the oFDC in modified release mode.

The oFDC of the present invention may comprise both LEV and LCM in a matrix which further comprise at least one agent which delays the release of LEV and LCM from said matrix (such agent, a “matrix retarding agent”). The matrix retarding agent(s) may be present in an amount of at least about 1 wt %, at least 1.5 wt %, at least about 2 wt %, at least 3 wt %, at least 4 wt %, at least 5 wt %, at least 6 wt %, at least 7 wt %, at least 8 wt %, 9 wt %, at least 10 wt %, at least 12 wt % or at least about 15 wt %, relative to the total weight of the formulation. In order to limit the size of the oFDC as much as possible, the matrix retarding agent(s) should be present in the matrix in an amount of less than 50 wt %, preferably less than 45 wt %, or at the most 40 wt %, at the most 35 wt %, or even more preferably at the most 30 wt %, or less, relative to the total weight of the formulation. In particular, the matrix retardation agent(s) may be present in the matrix in an overall amount of between about 10 wt % to 45 wt %, preferably 10 wt % to 40 wt %, more preferably 15 wt % to 35 wt %, even more preferably up to 30 wt % relative to the total weight of the formulation.

The matrix retardation agent may be selected from polymeric and non-polymeric matrix retardation agents.

Examples for suitable release modifying agents, and suitable drug release profiles can be taken from WO 2012/084126, WO 2012/072556, and from WO 2006/080029. Suitable release modifying agents in the matrix may include hydrophilic polymers (such as e.g. poloxamers, hydroxyethylcellulose, hydroxypropylcellulose (HPC), methylcellulose, carboxymethylcellulose, hydroxypropylmethylcellulose (HPMC), polyvinyl pyrrolidone, polyvinyl alcohols, modified starch, pregelatinized starch, hydroxypropyl starch, sodium hyaluronate, alginic acid, alginate salts, carrageenan, chitosan, guar gum, pectin, xanthan gum, and the like), hydrophobic polymers or non-polymeric substances (such as e.g. C8-C30 monohydric alcohols, monoglycerides, diglycerides, triglycerides, glycerine esters, hydrogenated castor oil, glyceryl behenate, hydrogenated soybean oil, lauroyl macrogolglycerides, stearyl macrogolglycerides, glyceryl palmitostearate, cethyl palmitate, glycerol esters of fatty acids and cetyl alcohol and the like), and inert polymers (such as acrylic resins, cellulose derivatives, vinyl acetate derivatives, and non-water soluble polyesters, preferably selected from the group of polyvinyl acetate, ethylcellulose, hydroxypropylmethylcellulose acetate phthalate, hydroxypropylmethylcellulose acetate succinate, shellac, poly-methacrylic acid derivatives, methacrylic acid copolymer type A, methacrylic acid copolymer type B, methacrylic acid copolymer type C, ammonio methacrylate copolymer type A, ammonio methacrylate copolymer type B, neutral ethyl methyl methacrylate copolymer, basic butylated methacrylate copolymer, and the like).

In one preferred aspect, the retardation agent is a hydrophilic matrix retardation agent. Hydrophilic retardation agents have the general advantages of usually becoming completely degraded in the animal body, being well characterized excipients, and showing good technical processability also on larger scale. It has also been shown in the present disclosure that hydrophilic matrix retardation agents are surprisingly well suited to control the dissolution of LCM and LEV from the same oFDC.

The hydrophilic matrix retardation agent may be selected from the group of gums, cellulose ethers, cellulose esters, and other cellulose derivatives, gelatine, polysaccharides, starch, starch derivatives, vinyl acetate and its derivatives, vinyl pyrrolidone and its derivatives, and polyethylene glycols. The hydrophilic matrix retardation agents are preferably selected from the group of poloxamers, hydroxyethylcellulose, hydroxypropylcellulose (HPC), methylcellulose, carboxymethylcellulose, hydroxypropylmethylcellulose (HPMC), polyvinyl pyrrolidone, polyvinyl alcohols, modified starch, pregelatinized starch, hydroxypropyl starch, sodium hyaluronate, alginic acid, alginate salts, carrageenan, chitosan, guar gum, pectin, and xanthan gum.

In one preferred aspect, the matrix retardation agent present in the oFDC is a hydrophilic polymer material selected from cellulose derivatives such as hydroxyethylcellulose, hydroxypropylcellulose (HPC), methylcellulose, and in particular hydroxypropylmethylcellulose (HPMC); such cellulose derivatives having a viscosity of about 50 mPa·s to 200,000 mPa·s in a 2 wt % aqueous solution at 20° C., preferably a viscosity of about 80 mPa·s to about 50,000 mPa·s in a 2 wt % aqueous solution at 20° C. or between about 100 mPa·s and about 25,000 mPa·s, wherein any viscosity referred to in this application is determined by Ubbelohde or Ostwald capillary according to the USP (Edition 24) method <911>. “Viscosity” as used herein is sometimes also termed “apparent viscosity” in the art.

Preferred matrix retarding agents are hydrophilic polymers with a medium viscosity between about 1000 and 25000 mPa·s. Particularly preferred matrix retarding agents are HPMC qualities with a medium viscosity between about 3000 and 25000 mPa·s; such “medium viscosity HPMCs” are commercially available from e.g. Dow Corning under the brand names K4M Premium CR®, E4M Premium CR®, E10M Premium CR® or K15Premium CR®, having viscosities of about 3000 and 6000 mPa·s, about 7500 and 14000 mPa·s, and between about 10000 and 21000 mPa·s, respectively. These medium viscosity HPMCs may be used as sole matrix retardation agents, or may be used in admixture with other hydrophilic polymers having a similar or lower viscosity. If used as sole matrix retarding agent, they may be typically used in amounts of 15 to 30 wt % relative to the total weight of the formulation.

If a high viscosity hydrophilic polymer, in particular a cellulose derivative, e.g. HPC or HPMC, having a viscosity of at least about 30,000 mPa·s, preferably of at least about 50,000 Pa·s or at least about 100,000 mPa·s in 2% aqueous solution is being used as retarding agent, the amount of HPMC in the formulation can surprisingly be as low as about 8 wt % or less, 6 wt % or less, 5 wt % or less, 4 wt % or less, 3 wt % or less or even between 1 wt % and 2 wt % relative to the total weight of the formulation.

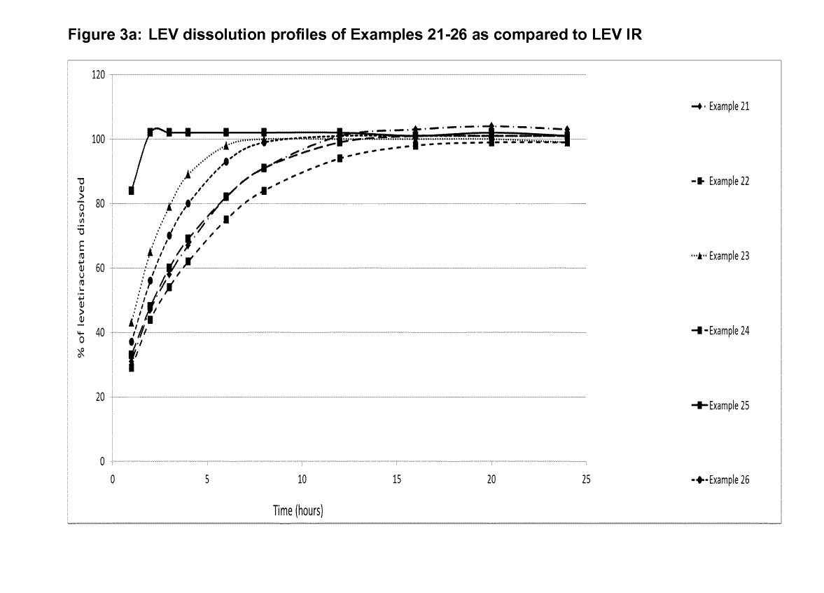

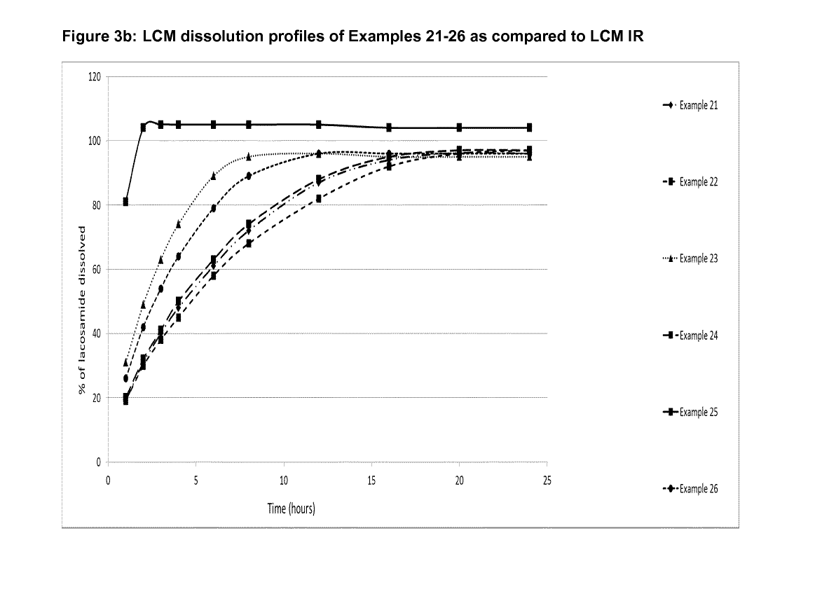

In addition to the medium to high-viscosity cellulose derivatives described above, it has been surprisingly found that cellulose derivatives with a low viscosity are also well suited for the retardation of LCM and LEV, see in particular Example 26 herein. This is particularly unexpected in view of the high water solubilities of LCM and LEV. It has been found by the present inventors that cellulose derivatives such as e.g. HPMC with a viscosity as low as 50-1000 mPa·s, or even as low as 80-120 mPa·s in a 2 wt-% aqueous solution at 20° C., can be used to effectively modify the release of both drugs from the oFDC (see example 26 and FIGS. 3a and 3b). If a low viscosity cellulose derivative is being used as the only matrix retarding agent, the concentrations of the cellulose derivative may be at least 20 wt %, or 30 wt % relative to the total weight of the formulation.

In a preferred embodiment, a medium viscosity hydrophilic polymer, preferably HPMC, with a viscosity of about 1000 and 25000 mPa·s, preferably between about 3000 and 25000 mPa·s, and a low viscosity hydrophilic polymer, such as e.g. a HPMC having a viscosity of between about 50 and 1000 mPa·s, or between about 80 and 120 mPa·s can be advantageously used in admixture. A suitable low viscosity HPMC is commercially available from e.g. Dow Corning under the brand name K100LV Premium®. In this embodiment, without wished to be bound to any theory, the low viscosity polymer, e.g. the HPMC, is thought to modulate or fine tune the stronger retarding effect of the medium viscosity hydrophilic polymer. Suitable mixtures are for example those containing (a) between 5 and 30 wt % of a medium viscosity HPMC with a medium viscosity between about 3000 and 25000 mPa·s (such as e.g. K15M CR, Dow Corning) and (b) between 10 and 30 wt % of a low viscosity HPMC having a viscosity of between about 50 and 1000 mPa·s (such as e.g. K100 LV CR/Dow Corning), Preferably the total amount of the low viscosity and the medium viscosity HPMC is no more than 40 wt % preferably, no more than 35 wt % or 30 wt %. Examples are given in table 13, see Examples 21 and 22, below.

Accordingly, in one aspect the composition of the oFDC comprises a matrix comprising
(a) at least 55 wt %, preferably at least 60 wt %, more preferably at least 65 wt %, even more preferably at least 70 wt % of active ingredient consisting of levetiracetam and lacosamide in a ratio (wt/wt) of about 2:1 to about 20:1, or of about 3:1 to about 20:1, preferably of about 3:1 to about 15:1, or between about 5:1 to about 15:1, and
(b) up to 45 wt %, preferably up to 40 wt %, more preferably up to 35 wt % or up to 30 wt % of excipients, which comprise
   (b1) 0.5-2 wt %, preferably 0.75-1.25 wt % colloidal silicon dioxide,
   (b2) 2-8 wt %, preferably 2-5 wt % crospovidone,
   (b3) 0.75-2.5 wt %, preferably 0.75-2 wt % or 1-2 wt %, more preferably 1-1.5 wt % of a hydrophilic lubricant, preferably sodium stearylfumarate, and
   (b4) up to 40 wt %, preferably at the most 35 wt %, more preferably at the most 30 wt % of at least one matrix retarding agent, which is/are preferably selected from the group of hydrophilic polymers as described herein, and is/are particularly preferable a medium viscosity HPMC, or a mixture of medium and low viscosity HPMCs, such as for example a mixture of (a) between 5 and 30 wt % of a HPMC with a medium viscosity of between about 3000 and 25000 mPa·s (such as e.g. K15M CR, Dow Corning) and (b) between 10 and 30 wt % of a HPMC having a low viscosity of between about 50 and 1000 mPa·s,
      wherein such composition preferably releases both, LCM and LEV, in a modified release fashion.

In another aspect, the matrix retardation agent of the oFDC is a polyethylene glycol having a viscosity given as a 1% solution in water at 25° C. of between about 1,000 and 50,000 mPa·s, preferably between 1,500 and 20,000 mPa·s (cP), and particularly preferable between about 1500 mPa·s and 15000 mPa·s.

In just another aspect, the matrix retardation agent of the oFDC is a starch having a viscosity given as a 2% solution in water at 25° C. of between about 20 and 200 mPa·s when measured using Ubbelohde or Ostwald capillary viscosity, preferably between 50 and 100 mPa·s (cP), and particularly preferably of about 70 mPa·s.

In just another aspect, the matrix retardation agent of the oFDC is xanthan having a viscosity given as a 1% solution in water at 25° C. of between about 500 and 2000 mPa·s when measured using Ubbelohde capillary viscosity, preferably between 1000 and 2000 mPa·s (cP).

In another preferred aspect, the oFDC comprises a matrix core comprising LEV and LCM in substantially non-delayed, i.e. immediate release fashion, which core is surrounded by a functional coating which delays the release of both LCM, and LEV from the oFDC. In this embodiment, the core may have an immediate release composition as described herein (see Examples 1-13), i.e. the drug load of the core can be as high as described previously, while any release modifying agents are present only in the coating, and maybe as low as below 10 wt %, or below 5 wt %, or even below 4 wt %, or below 3 wt % of the weight of the total composition.

If the oFDC comprises a release controlling layer, this layer may comprise a water-insoluble wax or at least one polymer capable of delaying the release of LEV and/or LCM. For example, the release controlling layer may comprise at least one release delaying polymer which is selected from acrylic resins, cellulose derivatives, or vinyl acetate derivatives. These polymers may be water-soluble or water-insoluble. These polymers are preferably selected from polyvinyl pyrrolidone, polyvinyl acetate, ethylcellulose, hydroxypropylmethylcellulose acetate phthalate, hydroxypropylcellulose, hydroxypropylmethylcellulose acetate succinate, shellac, methacrylate-containing copolymers (including e.g. methacrylic acid copolymer type A, methacrylic acid copolymer type B, methacrylic acid copolymer type C, ammonio methacrylate copolymer type A, ammonio methacrylate copolymer type B, basic butylated methacrylate copolymer, and poly(ethyl acrylate-co-methyl methacrylate)copolymers). Alternatively, water-soluble pore-forming agents may be present in the release controlling layer as well. Water-soluble pore-forming agents such as hydroxypropylmethylcellulose, polyethyleneglycol, mono- or disaccharides, and inorganic salts may be embedded within the less soluble release controlling agent(s) and rapidly dissolve in aqueous environment thus opening pores through which LCM and/or LEV are released.

Preferred release delaying polymers in the coating are selected from ethylcellulose, (e.g. SURELEASE® ETHYLCELLULOSE dispersion type B NF E-7-19040 provided by Colorcon/US), polyvinylacetate (e.g. Kollicoat SR 30D® provided by BASF/Germany), and poly(ethyl acrylate-co-methyl methacrylate)copolymers (e.g. Eudragit NE30D® obtainable from Evonik Industries/Germany).

For example, a polymer-based functional coating may be applied to the lacosamide matrix core as an aqueous dispersion which also contains plasticizer and stabilizer. A typical example for such a coating is a dispersion consisting of:
(a) 15-30 wt % polymer such as e.g. ethylcellulose (preferably having a viscosity of 10-50 cP, e.g. 20 cP)
(b) 4-6 wt % ammonium hydroxide (about 30% aqueous solution)
(c) 4-5 wt % medium chain triglycerides
(d) 2-3 wt % oleic acid
(e) at 100 wt % purified water

In an alternative embodiment, LEV and LCM are comprised in different layers/matrices of the inventive oFDC. For example, LEV may be embedded in an inner layer/matrix which may be surrounded by an outer layer encompassing LCM, or vice versa. In the case of such a bilayer tablet, both layers may release the respective drug in immediate release or both layers release the respective drugs in modified release fashion, i.e. in essentially the same or in similar dissolution rates. In one embodiment, LEV and LCM may be released from the different layers in different rates, such that e.g. LEV may be released in substantially immediate release dissolution rates whereas LCM dissolves in a modified or delayed release fashion. Likewise, LCM may be embedded in an immediate release outer layer, whereas LEV may be provided in a modified release fashion from an inner matrix comprising release modifying agents. This differentiated release profile may have the advantage that the peaks of the maximum and minimal drug concentrations of both drugs in the patient (Cmax, Cmin, respectively) do not appear simultaneously, and as a consequence, neither the anticonvulsant effects nor the unwanted effects of both drugs may occur at the same time but are spread over the administration interval. Also, such differing release rates may be used to adapt the oFDC to the lower half-life of LEV compared to LCM, e.g. by delaying the release of LEV while releasing LCM in an immediate release fashion, or less delayed than LEV.

Alternatively, LEV and LCM may be formulated in a multiplicity of MUDs, such as minitablets or granules with the same or with different release profile. For example, LEV and LCM may both be contained in the same matrix of the MUD thus showing the same or very similar release rates. In another embodiment, LEV may be formulated into an immediate release layer or segment of the minitablet, while LCM may be formulated into a delayed or modified release layer or segment of the same minitablets, or vice versa.

In just another embodiment, LCM and LEV may both be incorporated in a first set of delayed or modified release minitablets with a different release rate compared to a second part of minitablets which provide both LEV and LCM in immediate release fashion. Both sets of the LEV and LCM-containing minitablets may be packed in a joint capsule or sachet for combined administration to achieve a pulsed release profile of both drugs.

Accordingly, one embodiment of the present invention relates to an oFDC comprising multiple unit dosage forms comprising pellets, minitablets, and granules which comprise LCM and LEV as active ingredients. Preferably the MUD units on average have a drug load (i.e. a content of LEV and LCM) of more than 75 wt %, more than 80 wt %, more than 85 wt %, more than 90 wt %, more than 93 wt %, or more than 95 wt %, relative to the total weight of the units. In one embodiment, LEV and LCM may both be released from the MUDs, e.g. from pellets granules or minitablets, in immediate release fashion. In another embodiment, LEV and LCM may be released from the MUDs in modified release fashion. In just another embodiment, LEV may be released from the MUD in modified release fashion while LCM is released in IR fashion. In just another embodiment, LCM is released from the MUDs in modified release fashion while LEV is released in IR course.

If LEV and LCM are both released from the oFDC in a modified/delayed release fashion, the oFDC may be administered twice a day or once a day. If the oFDCs are administered only once a day, the dosing regimens as described hereinbefore are correspondingly adapted. For example, the improved dosing regimen may comprise an oral fixed dose combination of levetiracetam and lacosamide for use as a medicine, preferably for use in the prophylaxis, alleviation and/or treatment of epileptogenesis, of an epileptic disorder and/or of epileptic seizures, which comprises the once daily administration of one or two entities of said fixed dosage combination at a given time (such as e.g. one or two tablets or capsules in the morning), wherein one entity provides the combined release of lacosamide and levetiracetam in dosages selected from e.g. (a) 50 mg LCM+250 mg LEV, (b) 50 mg LCM+500 mg LEV, (c) 50 mg LCM+750 mg LEV, (d) 100 mg LCM+500 mg LEV, (e) 100 mg LCM+750 mg LEV, and (f) 200 mg LCM+1000 mg LEV, and wherein said entity preferably releases LCM and LEV in a modified release fashion, as described further above, wherein the Tmax of at least one or of both compounds are at least 3 hours, at least 4 hours or more. Other suitable dosages for the once daily administration regimen are (g) 150 mg LCM+500 mg LEV, (h) 150 mg LCM+1000 mg LEV. Particularly preferred dosages for use in the once daily dosing regimen are (d) 100 mg LCM+500 mg LEV, (j) 100 mg LCM+1000 mg LEV, (k) 100 mg LCM+1500 mg LEV, (i) 200 mg LCM+500 mg LEV, (f) LEV 200 mg LCM+1000 mg LEV, (l) 200 mg LCM+1500 mg LEV, (m) 300 mg LCM+500 mg LEV, (n) 300 mg LCM+1000 mg LEV, (o) 400 mg LCM+500 mg LEV, and (p) 400 mg LCM+1000 mg LEV. As a result, in these particular dosing regimens only one or two oFDC entities need to be swallowed by the patient per day. Such treatment regimens may reduce the pill burden compared to the twice daily LEV+LCM treatment regimen of the individual LEV and LCM formulations known in the art (2 Vimpat®+2 Keppra® IR tablets, or 2 Vimpat®+1 Keppra® XR, extended release tablet) by 66% to 75%. In a preferred embodiment, the oFDC entity for a once-a-day treatment is a single unit dosage form and particularly preferable a tablet.

One aspect of the present invention relates to a method of preventing, alleviating and/or treating of an epileptic disorder and/or of epileptic seizures, or of epileptogenesis, comprising administering to a patient in need thereof an oral fixed dose combination of levetiracetam and lacosamide, wherein said administration comprises the once administration of one or two entities of said fixed dosage combination (such as e.g. one tablet or capsule), wherein one entity provides the combined release of lacosamide and levetiracetam in dosages selected from e.g. (a) 50 mg LCM+250 mg LEV, (b) 50 mg LCM+500 mg LEV, (c) 50 mg LCM+750 mg LEV, (d) 100 mg LCM+500 mg LEV, (e) 100 mg LCM+750 mg LEV, and (f) 200 mg LCM+1000 mg LEV. Other suitable dosages for use in this method including a once-a-day FDC administration are for example (g) 150 mg LCM+500 mg LEV, (h) 150 mg LCM+1000 mg LEV, and in particular (d) 100 mg LCM+500 mg LEV, (j) 100 mg LCM+1000 mg LEV, (k) 100 mg LCM+1500 mg LEV, (i) 200 mg LCM+500 mg LEV, (f) LEV 200 mg LCM+1000 mg LEV, (l) 200 mg LCM+1500 mg LEV, (m) 300 mg LCM+500 mg LEV, (n) 300 mg LCM+1000 mg LEV, (o) 400 mg LCM+500 mg LEV, and (p) 400 mg LCM+1000 mg LEV. In a preferred embodiment, the oFDC entity administered is a single unit dosage form and particularly preferable a tablet, and even more preferable a tablet which releases at least one, more preferably both of LEV and LCM in a delayed release fashion.

The pharmaceutical composition (oFDC) according to the present invention, including preferably the fixed dose tablets, comprises excipients in a total amount of up to 25 wt %, preferably up to 20 wt %, up to 15 wt %, up to 10 wt % (e.g. 3-10 wt %), preferably up to 7 wt % (e.g. 4-7 wt %), more preferably up to 5 wt %. Somewhat higher total amounts of excipients of up to 40 wt % but preferably at the most 30 wt % may be needed in some cases of modified release oFDCs, depending on the choice of the rate controlling principles and agents. This is particularly the case for oFDCs with modified release matrices, in which sometimes matrix retardation agents in amounts of more than 15 wt % are needed, depending on the choice of said agent. In contrast, oFDCs with functional coatings typically require amounts of release delaying agents below 10 wt %, or even below 5 wt %, i.e. the overall drug load may still be well above 90 wt %, see e.g. Example 27.

Excipients used in the pharmaceutical composition of the present disclosure are principally those known to a person skilled in the art. In particular, the oFDC may comprise glidants, disintegrants, lubricants, stabilizers, antioxidants, binders, fillers, release modifying polymers, coating material, and the like. Suitable excipients may be taken from standard books like e.g. Remington, The Science and Practice of Pharmacy, ed Lippincott, Williams and Wilkins, Baltimore US.

Preferably the amount of excipients is reduced as much as possible, as described herein, and preferably the excipients comprise (a) at least one gildant, (b) at least one disintegrant, (c) at least one lubricant, and optionally (d) a binder or diluent, and/or (e) a release modifying coating, and/or modified release agent in the matrix.

Hence, in one embodiment, the pharmaceutical composition (i.e. the oFDC) according to the invention comprises up to about 15 wt %, up to about 10 wt %, up to about 7 wt %, preferably up to about 5 wt % excipients, wherein the excipients basically consist of (a) at least one gildant, (b) at least one disintegrant, and (c) at least one lubricant, and optionally (d) a release modifying coating and/or modified release agent in the matrix.

In one aspect, the glidant is selected from magnesium silicate, magnesium trisilicate, sodium stearate, hydrophobic colloidal silica, magnesium oxide, talc, colloidal silicon dioxide, and is preferably colloidal silicon dioxide, most preferably in an amount of about 0.5-2 wt %. Non limiting examples of suitable qualities of colloidal silicon oxide are for example Aerosil 200, or Aerosil 300 (both available from Degussa Evonik).

In one aspect, the disintegrant is selected from croscarmellose, crospovidone, sodium starch glycolate, pregelatinized starch, native starch, and preferably is crospovidone, particularly in an amount of 2-8 wt %, or 2-5 wt %. Non limiting examples of suitable qualities of crospovidon are for example XL, INF-10 (available from ISP) or Kollidon CL (available from BASF).

In one aspect, the lubricant is selected from magnesium stearate, sodium stearyl fumarate, hydrogenated castor oil, zinc stearate, calcium stearate, sucrose stearate, glycerylpalmitoyl stearate, hydrogenated vegetable oil, potassium benzoate, leucine, polyethylene glycol, palmitic acid, and stearic acid, and is preferably a hydrophilic lubricant selected from sodium stearyl fumarate, sodium laurylsulfate, potassium benzoate, and polyethylene glycol, e.g. in amounts of about 0.75-2 wt %. In a particular embodiment, the hydrophilic lubricant is sodium stearylfumarate, preferably in an amount of about 0.75-2 wt %. If a non-hydrophilic lubricant such as magnesium stearate is used, the amounts needed are usually somewhat higher any may be at least 1.0 preferably at least 1.2 wt %.

The binder or diluent, if present, may be selected from agar, guar gum, copovidone, povidone, polyethylene glycol, polyethylene oxide, ethylcellulose, methylcellulose, hydroxypropylcellulose, hydroxyethylcellulose, hydroxypropylmethylcellulose, pregelatinized starch, anhydrous lactose and related hydrates, microcrystalline cellulose, powdered cellulose, calcium phosphate tribasic, anhydrous calcium dibasic phosphate and related hydrates, polyols (e.g. mannitol, maltitol, sorbitol), isomalt, sucrose, polydextrose, maltodextrin, dextrate and may be present in an amount of up to 20 wt %, 15 wt %, up to 10 wt %, preferably up to 7.5 wt %, or up to 5 wt %. In one embodiment, the oFDC does not contain any binder or diluent.

In one particular aspect of the invention, the amount of excipients and thus of the volume of the oFDC, preferably the tablet, is limited as far as possible.

In one aspect, the oral pharmaceutical composition (oFDC) is an immediate release composition, preferably a tablet releasing LEV and LCM in an immediate release fashion, comprising
(a) at least 90 wt %, preferably 90-97 wt %, more preferably at least 93 wt % such as 93-96 wt % or even more than 95 wt % of active ingredient, consisting of levetiracetam and lacosamide in a ratio (wt/wt) of about 2:1 to about 20:1, or of 3:1 to about 20:1, preferably of about 5:1 to about 15:1,
(b) up to 10 wt %, preferably up to 7-10 wt %, preferably up to 7 wt %, such as 4-7 wt %, or only up to 5 wt % of excipients, which comprises at least one disintegrant, preferably in an amount of 2-8 wt %, preferably 2-5 wt %, or 2-3 wt %. Said disintegrant is preferably selected from croscarmellose, crospovidone, sodium starch glycolate, pregelatinized starch, native starch, and preferably is crospovidone.

In a preferred aspect of the disclosure, this immediate release composition is surrounded by a non-functional coating.

Alternatively, the composition may be surrounded by a functional coating comprising a release controlling agent, wherein said functional coating is present in an amount of preferably less than 10 wt %, less than 7.5 wt %, or less than 5 wt %, and wherein said release controlling agent is present preferably in an amount of less than 10 wt %, more preferably less than 5 wt % or even less than 3 wt %, relative to the total weight of the oFDC.

It is to be understood that if to an immediate release oFDC described herein a functional coating is optionally applied, all amounts given herein in connection with said immediate release formulation refer to the IR “core” of the formulation only. If no coating is added to this IR formulation, these amounts also refer to the total weight of the formulation, whereas in case a functional coating is applied, the amounts of IR components relative to the total weight of the composition need to be recalculated. For example, in the embodiment described immediately before, the IR oFDC comprises at least 90 wt % drug and up to 10 wt % excipients relative to the total weight of the formulation. If a functional coating is applied to said immediate release oFDC “core” and the coating represents 10 wt % of the total formulation, the overall composition would be 90 wt % IR core (composed of 90 wt % drug and 10 wt % excipients) and 10 wt % functional coating, i.e. the oFDC would contain at least 81 wt % drug, up to 9 wt % matrix excipients and 10 wt % coating relative to the overall weight of the formulation. This correspondingly applies to the disclosures following below. Preferably, the overall drug load of the oFDC is still above 90 wt % even if a functional coating is present.

In one aspect, the pharmaceutical composition (oFDC), preferably a tablet, comprises
(a) at least 90 wt %, preferably 90-97 wt %, more preferably at least 93 wt %, such as 93-96 wt % or even more than 95 wt % of active ingredient, consisting of levetiracetam and lacosamide in a ratio (wt/wt) of about 2:1 to about 20:1, or of about 3:1 to about 20:1, preferably of about 3:1 to about 15:1, or between about 5:1 to about 15:1,
(b) up to 10 wt %, preferably up to 7-10 wt %, preferably up to 7 wt %, such as 4-7 wt %, or only up to 5 wt % of excipients, which comprises
   (b1) 0.5-2 wt %, preferably 0.75-1.25 wt %, such as e.g. about 1 wt % of a glidant, wherein the glidant is preferably selected from magnesium silicate, magnesium trisilicate, sodium stearate, hydrophobic colloidal silica, magnesium oxide, talc, colloidal silicon dioxide,
   (b2) 2-8 wt %, preferably 2-5 wt %, or 2-3 wt % of a disintegrant, wherein the disintegrant is preferably selected from croscarmellose, crospovidone, sodium starch glycolate, pregelatinized starch, native starch, and preferably is crospovidone,
   (b3) 0.75-2.5 wt %, preferably 0.75-2 wt %, more preferably 1-1.5 wt % such as e.g. about 1 wt % of a lubricant, which is preferably selected from magnesium stearate, sodium stearyl fumarate, hydrogenated castor oil, zinc stearate, calcium stearate, sucrose stearate, glycerylpalmitoyl stearate, hydrogenated vegetable oil, potassium benzoate, leucine, polyethylene glycol, palmitic acid, and stearic acid, and more preferably a hydrophilic lubricant selected from sodium stearyl fumarate, sodium laurylsulfate, potassium benzoate, and polyethylene glycol,
      wherein such composition preferably releases both, LCM and LEV, in an immediate release fashion. In one embodiment, this IR composition may be surrounded by a functional coating comprising a release controlling agent, wherein the functional coating is preferably applied in an amount of less than 10 wt %, more preferably less than 5 wt % relative to the total weight of the formulation, and wherein said release controlling agent is present preferably in an amount of less than 10 wt %, more preferably less than 5 wt % or even less than 3 wt %, relative to the total weight of the oFDC, and wherein the relative amounts of components of the IR composition hereinbefore refer to the IR core instead of the total weight of the composition.

In one aspect the pharmaceutical composition comprises

(a) at least 93 wt %, preferably at least 95% active ingredient consisting of levetiracetam and lacosamide in a ratio (wt/wt) of about 2:1 to about 20:1, or of about 3:1 to about 20:1, preferably of about 3:1 to about 15:1, or between about 5:1 to about 15:1,
(b) up to 7 wt %, preferably up to 5 wt % of excipients, which comprises
(b1) 0.5-2 wt %, preferably 0.75-1.25 wt % colloidal silicon dioxide,
(b2) 2-8 wt %, preferably 2-5 wt % crospovidone, and
(b3) 0.75-2.5 wt %, preferably 0.75-2 wt % or 1-2 wt %, more preferably 1-1.5 wt % of a hydrophilic lubricant, preferably sodium stearylfumarate,
   and wherein such composition preferably releases both, LCM and LEV, in an immediate release fashion.

In one aspect, this before mentioned composition is a modified/delayed core to which a release modifying functional coating is added, wherein the functional coating is preferably applied in an amount of less than 10 wt %, more preferably less than 5 wt % relative to the total weight of the formulation, and wherein said release controlling agent is present preferably in an amount of less than 10 wt %, more preferably less than 5 wt % or even less than 3 wt %, relative to the total weight of the oFDC, and wherein the relative amounts of components of the IR composition hereinbefore refer to the IR core instead of the total weight of the composition.

In one aspect, the pharmaceutical composition is a tablet, preferably an immediate release tablet, which comprises an immediate release matrix comprising
(a) at least 93 wt %, preferably at least 95% active ingredient consisting of levetiracetam and lacosamide in a ratio (wt/wt) of about 2:1 to about 20:1, or of about 3:1 to about 20:1, preferably of about 3:1 to about 15:1, or between about 5:1 to about 15:1, wherein
   (a1) the amount of LCM is from 50-400 mg and is preferably selected from 50 mg, 100 mg, 150 mg and 200 mg, and
   (a2) wherein the amount of LEV is from 250-1500 mg, is preferably selected from 250 mg, 500 mg, 750 mg and 1000 mg, and is particularly preferably selected from 250 and 500 mg; and
(b) up to 7 wt %, preferably up to 5 wt % of excipients, comprising
   (b1) 0.5-2 wt %, preferably 0.75-1.25 wt % colloidal silicone dioxide; and
   (b2) 2-5 wt %, preferably 2-3 wt % crospovidone, and
   (b3) 0.75-2.5 wt %, preferably 1-2 wt %, more preferably 1-1.5 wt % of a hydrophilic lubricant, preferably sodium stearylfumarate.

In one aspect, this composition is a modified/delayed composition to which an outer coating is added, wherein the functional coating is preferably applied in an amount of less than 10 wt %, more preferably less than 7.5 wt % or even less than 5 wt % relative to the total weight of the formulation, and wherein said release controlling agent is present preferably in an amount of less than 10 wt %, more preferably less than 5 wt % or even less than 3 wt %, relative to the total weight of the oFDC and wherein the relative amounts of components of the IR composition hereinbefore refer to the IR core instead of the total weight of the composition.

In one specific aspect the pharmaceutical composition is a tablet, preferably an immediate release tablet, which comprises a matrix essentially consisting of
(a) at least 93 wt %, preferably at least 95% active ingredient consisting of levetiracetam and lacosamide in a ratio (wt/wt) of about 3:1 to about 15:1, or of about 5:1 to about 15:1, wherein
   (a1) the amount of LCM is selected from 50 mg, 100 mg, 150 mg, and 200 mg, and
   (a2) wherein the amount of LEV is selected from 250 mg, 500 mg, and 750 mg; and
(b) up to 7 wt %, preferably up to 5 wt % of excipients, comprising
   (b1) 0.75-1.25 wt % colloidal silicon dioxide and
   (b2) 2-3 wt % crospovidone, and
   (b3) 1-1.5 wt % of a hydrophilic lubricant, preferably sodium stearylfumarate.

In one aspect, this composition is a modified/delayed composition to which an outer coating is added, wherein the functional coating is preferably applied in an amount of less than 10 wt %, more preferably less than 7.5 wt % or even less than 5 wt % relative to the total weight of the formulation, and wherein said release controlling agent is present preferably in an amount of less than 10 wt %, more preferably less than 5 wt % or even less than 3 wt %, relative to the total weight of the oFDC and wherein the relative amounts of components of the IR composition hereinbefore refer to the IR core instead of the total weight of the composition.

Exemplary oFDC formulations with a drug load of 95.3 wt % are given in Table 3 below:

TABLE 3

| | Ex-1 | Ex-2 | Ex-3 | Ex-4 | Ex-5 | Ex-6 | Ex-a | Ex-b |
|---|---|---|---|---|---|---|---|---|
| Leviteracetam (mg) | 500 | 500 | 750 | 750 | 1000 | 250 | 500 | 500 |
| Lacosamide (mg) | 50 | 100 | 50 | 100 | 200 | 50 | 150 | 200 |
| Colloidal silica 200 (%) | 1.00 | 1.00 | 1.00 | 1.00 | 1.00 | 1.00 | 1.00 | 1.00 |
| Crospovidone (%) | 2.50 | 2.50 | 2.50 | 2.50 | 2.50 | 2.50 | 2.50 | 2.50 |
| Sodium Stearyl | 1.20 | 1.20 | 1.20 | 1.20 | 1.20 | 1.20 | 1.20 | 1.20 |
| Fumarate (SSF) (%) | | | | | | | | |

Optionally an outer coating layer can be applied to the formulations described above. In one embodiment, the coating layer comprises a release controlling agent, and the coating layer is applied in an amount of preferably less than 10 wt %, more preferably less than 7.5 wt % or even less than 5 wt %, and the release controlling agent is preferably present in an amount of less than 10 wt %, more preferably less than 5 wt %, or even less than 3 wt %, relative to the overall weight of the oFDC, and wherein the relative amounts of components of the IR composition in Table 3 refer to the IR core instead of the total weight of the composition.

One aspect of the present invention is an oral pharmaceutical composition as further described herein, wherein the total volume of said composition is less than the combined total volume of the commercially available Vimpat® and Keppra® formulations at the time of invention, containing the same amounts of LCM and LEV, which volumes are given in the following Table 4:

| Commercial Formulation | Drug Load/Dosage | Volume (mm³) |
|---|---|---|
| Vimpat ® | 50 mg LCM | X1 = 99.80 |
| | 100 mg LCM | X2 = 199.59 |
| | 150 mg LCM | X3 = 299.39 |
| | 200 mg LCM | X4 = 399.18 |
| Keppra ® | 250 mg LEV | Y1 = 234.50 |
| | 500 mg LEV | Y2 = 467.19 |
| | 750 mg LEV | Y3 = 701.24 |
| | 1000 mg LEV | Y4 = 949.73 |

By way of example, an oFDC according to the present invention which contains 100 mg LCM and 500 mg of LEV shall preferably have a volume which is less than the combined volume of the Vimpat® and Keppra® tablets having the same drug content, i.e. less than X2 ml+Y2 ml=199.59 ml+467.19 ml=less than 666.78 ml.

Even more preferred, the volume of the oFDC should be more than 5%, preferably more than more than 10%, more preferably more than 15%, or even about 20% smaller than the combined volume of the corresponding Vimpat and Keppra tablets having the same dosage.

Exemplary volumes of oFDCs and volume reductions reached with the Examples 1-5 of the present invention compared to the corresponding commercial products in Table 4 are given in Table 5:

| | oFDC | Combined volume of commercial | % volume |
|---|---|---|---|
| Doses | volumes | Keppra ® and Vimpat ® | reduction |
| LEV/LCM | (mm³) | formulations | by oFDC |
| 500/50 | 547.00 | 99.80 + 467.19 = 566.99 | 3.53 |
| 500/100 | 555.06 | 199.59 + 467.19 = 666.78 | 16.76 |
| 750/50 | 727.18 | 99.80 + 701.24 = 801.04 | 9.22 |
| 750/100 | 777.18 | 199.59 + 701.24 = 900.83 | 13.73 |
| 1000/200 | 1088.66 | 949.72 + 399.18 = 1348.90 | 19.29 |

It has been found, surprisingly, that despite the minimized amount of excipients the pharmaceutical compositions show robust dissolution profiles and good physical stability, as shown in the examples.

### Injectable Solutions

Another aspect of the present invention is a fixed dose combination for injection and/or infusion (“iFDC”) comprising both, LEV and LCM. Such an iFDC comprises LCM in 50 mg, 100 mg, 150 mg, 200 mg or 400 mg; typical amounts of LEV are 250 mg, 500 mg, 750 mg, 1000 mg or 1500 mg. Said iFDC is either a solution which is ready for use, i.e. for immediate parenteral administration, or is a concentrated solution or maybe a solid, dry mixture such as e.g. powder or lyophilisate comprising both drugs and optional excipients, which dry mixture is dissolved prior to injection or infusion.

Preferably the administration of LCM and LEV from the iFDC takes place simultaneously, i.e. during injection or infusion both drugs are administered as a mixture in respective amounts over time which correspond to the ratio LCM:LEV in the iFDC. Typically, the iFDCs of the present disclosure are immediate release iFDCs which rapidly release LEV and LCM into the patient's circulation. Such an iFDC is particularly suitable in emergency situations such as e.g. severe generalized tonic-clonic seizures, acute repetitive seizures or status epilepticus, in particular if these were already resistant to other anticonvulsants. Also, the iFDCs may be useful as i.v. loading dose to rapidly provide appropriate plasma levels of LCM-LEV to a patient before switching to oral medication.

Due to the excellent physicochemical compatibility between LCM and LEV, inventors found that an iFDC with a rather low volume of a concentrated solution, or preferably, an iFDC which is based on a substantially dry mixture of both compounds can be provided which can conveniently be stored in a suitably small container, such as e.g. in a syringe, a vial, or an infusion bag, and which can rapidly be diluted or dissolved, respectively, using suitable isotonic buffer solutions.

Hence, in one aspect of the invention, the iFDC comprising LCM and LEV is a concentrated solution which may need to be diluted before administration by a factor of e.g. 1:5, 1:10. 1:20. 1:30, or 1:50. In one preferred aspect, the iFDC comprising LCM and LEV is a dry mixture comprising both compounds which need to be dissolved with an isotonic solution of a suitable volume before administration to a patient.

Typical amounts of LCM in the iFDC are 50 mg, 100 mg, 150 mg, 200 mg or 400 mg; typical amounts of LEV are 250 mg, 500 mg, 750 mg, 1000 mg or 1500 mg. Typical volumes for infusion or injection are between 50 and 500 ml, preferably about 50-150 ml, more preferably about 100 ml. Typical ratios of LEV:LCM (wt/wt) are from about 1:1 to about 20:1, preferably from 2:1 to about 20:1, and more preferably from about 3:1 to about 15:1, or between 5:1 and 15:1.

While the concentrated solution or the dry mixture containing LEV and LCM will preferably be stored in vials syringes, or infusion bags, the dilution to the final infusion volume may typically take place in an infusion bag. Before dissolving a dry mixture of LCM and LEV with the final administration volume, a more concentrated (“intermediate”) solution may be prepared in a 1^{st} step, for example in the vial or a syringe, which concentrated solution may then be transferred to the infusion bag for final dilution and administration to the patient. For example, a dry mixture comprising 200 mg LCM and 1000 mg LEV may be dissolved in 10-20 ml of a suitable solvent in a first step which then may be further diluted shortly prior to use to a typical infusion volume of 50-500 ml, preferably to about 50-150 ml, more preferably to about 100 ml.

Typical administration times of the parenteral administration are between 5 and 20 minutes, more preferably 10-15 minutes per administration, twice daily.

The following Table 6 illustrates non-limiting example iFDCs:

| | Exemplary volumes of a | |
|---|---|---|
| Doses | concentrated iFDC, or of an | Typical infusion |
| LEV/LCM | “intermediate” solution | volume |
| 250/50 | 5 ml | 50-100 ml |
| 250/100 | 5-10 ml | 100 ml |
| 500/100 | 5-10 ml | 100 ml |
| 500/150 | 5-15 ml | 100 ml |
| 500/200 | 5-20 ml | 100 ml |
| 750/150 | 7.5-15 ml | 100 ml |
| 750/200 | 7.5-20 ml | 100 ml |
| 1000/100 | 10 ml | 100 ml |
| 1000/200 | 10-20 ml | 100 ml |
| 1000/400 | 10-40 ml | 100 ml |

Suitable liquid phases used in the concentrated solutions are aqueous sodium chloride solutions and/or physiological buffers like e.g. phosphate, citrate or acetate buffers which are known to those skilled in the art. Prior to use, the dry mixture may be dissolved, or the concentrated iFDC may be diluted, respectively, with isotonic buffer such as e.g. 0.9% sodium chloride solution, lactated Ringer solution, 5% dextrose solution, or the like, preferably buffered to physiological pH at the final administration volume.

Accordingly, one aspect of the present invention is an iFDC comprising (a) LCM in an amount of 50 mg-400 mg, and preferably in an amount selected from 50 mg, 100 mg, 150 mg and 200 mg and, (b) LEV in an amount of 250-1500 mg, preferably selected from among 250 mg, 500 mg, 750 mg and 1000 mg, for use as a medicine, wherein said use comprises parenteral administration of said FDC, preferably by twice daily infusion or injection. In a preferred aspect, the parenteral administration is by injection or by infusion, and particularly preferable by infusion. In a preferred embodiment the injectable or infusible FDC (iFDC) is produced from an essentially dry mixture containing LEV and LCM, or from a concentrated solution with a concentration of LEV of between 2.5 and 10 wt %, and of LCM of between 1 and 5 wt %, preferably of between about 1 and 2 wt %, which may require dilution prior to its administration, preferably by infusion. The iFDC is preferably for use in the prophylaxis, alleviation and/or treatment of epileptogenesis of an epileptic disorder and/or of epileptic seizures.

One aspect of the present invention relates to an FDC comprising (a) LCM in an amount selected from 50 mg, 100 mg, 150 mg and 200 mg, as an essentially dry mixture containing LEV and LCM, or as a concentrated solution with a drug load of between 1 and 5 wt %, preferably of between 1 and 2 wt %, and (b) LEV in an amount selected from 250 mg, 500 mg, 750 mg and 1000 mg, as an essentially dry mixture containing LEV and LCM, or as a concentrated solution with a drug load of between 2.5 and 10 wt %, and preferably between 5 and 10 wt %, for use as a medicine, preferably for use in the prophylaxis, alleviation and/or treatment of epileptogenesis, of an epileptic disorder and/or of epileptic seizures, in particular in an emergency situation as described further above, or as loading dose for an oFDC, wherein said use includes the dilution of said FDC to a final volume of between 50 and 500 ml, preferably about 50-150 ml, more preferably to about 100 ml, within 48 hours, preferably within 24 hours, more preferably within 12, even more preferably within 6, 5, 4, 3, 2 or only 1 hour(s) before administration of the FDC to the patient by infusion. Alternatively In a particularly preferred option, the final iFDC is prepared from a concentrated solution or from a solid mixture within 3, within 2 or within 1 hour(s) before use or even immediately before administration to the patient. In one embodiment, the infusion of the iFDC to the patient will take place in a period of time of between 10 and 20 minutes, preferably in about 15 minutes.

In an alternative embodiment, the iFDC may contain a ready-for-use solution comprising LEV and LCM in a buffer solution at a volume which is ready for infusion, typically between 50 and 500 ml, preferably about 50-150 ml, more preferably about 100 ml. Such a ready-to-use iFDC may be advantageously stored in an infusion bag.

In just another embodiment, the iFDC may be prepared from ready-for-use solutions comprising LEV and LCM, respectively, each in a separate buffer solution at a volume which is ready for infusion, typically between 50 and 500 ml, preferably between about 50 and 150 ml, more preferably between about 50 ml or 100 ml. Both ready-to-use solutions can be either mixed immediately before administration, or they can be jointly administered via e.g. a joint tube. In one embodiment, both ready to use solutions are stored in a multi-chamber bag. For example, a two-chamber bag may be used for the storage of a ready-to-use solution of LEV in the 1^{st} chamber and a ready-to-use solution of LCM in the second chamber, and shortly before administration the content of the two chambers are mixed together.

Typical amounts of LCM in the ready-to-use iFDC or in the separate ready-to-use solutions for preparation of the iFDC are 50 mg, 100 mg, 150 mg, 200 mg or 400 mg; typical amounts of LEV are 250 mg, 500 mg, 750 mg, 1000 mg or 1500 mg. Typical buffers are acetate, phosphate, citrate, carbonate, which may or may not include stabilizers, or preservatives such as benzylic acid, phenol, parabens, chlorobutanol, chlorocresol, benzalkonium chloride, thimerosal, phenoxyethanol, if needed.

In another embodiment, the iFDC may be administered using a three chamber bag, comprising (a) a first chamber containing a concentrated storage solution of LEV, (b) a second chamber containing a concentrated solution of LCM, and (c) a third chamber containing an isotonic buffer, as described further above, for dilution of the mixture of the content of all chambers to the final administration volume. Typical amounts of LCM in the concentrated LCM solution are between 50 and 600 mg, more preferably between 50 and 400 mg, and in particular 50 mg, 100 mg, 150 mg or 200 mg. Typical amounts of LEV in the concentrated LEV solutions are between 100 and 3000 mg, preferably between 200 and 1500 mg and in particular 250 mg, 500 mg, 750 mg, 1000 mg or 1500 mg. Typical volumes of the concentrated solutions are between 1 and 20 ml, preferably between about 1 and 10 ml for the LCM solution and between 5 and 20 ml for the LEV solution. The volume of the isotonic buffer in the 3^{rd} chamber can be adjusted to provide a final administration volume of typically between 50 and 500 ml, and preferable between about 100 and 150 ml. Suitable liquid phases used in the concentrated solutions in chambers (a) and (b) are aqueous sodium chloride solutions and/or physiological buffers like acetate, phosphate, citrate, or carbonate buffers which may or may not include stabilizers, or preservatives such as benzylic acid, phenol, parabens, chlorobutanol, chlorocresol, benzalkonium chloride, thimerosal, phenoxyethanol, if needed, while the isotonic buffer in (c) may be selected from e.g. 0.9% sodium chloride solution, lactated Ringer solution, 5% dextrose solution, or the like, buffered to physiological pH.

Hence, one aspect of the present invention is a kit comprising (i) a multichamber infusion bag and (ii) the components of an FDC contained in said infusion bag, wherein the FDC comprises (a) a first chamber containing a LCM solution comprising LCM in an amount between about 50 mg and 600 mg, preferably between 50 mg and 400 mg, and more preferably in an amount selected from 50 mg, 100 mg, 150 mg and 200 mg, and (b) a second chamber containing a LEV solution comprising LEV in an amount between about 100 mg and 3000 mg, preferably between about 200 mg and 1500 mg, and more preferably in an amount selected from 250 mg, 500 mg, 750 mg, 1000 mg, and optionally (c) a third chamber comprising an isotonic buffer for dilution of the mixture of the LCM solution and the LEV solution shortly prior to administration. In one aspect, said kit is for use as a medicine, preferably for use in the prophylaxis, alleviation and/or treatment of epileptogenesis, of an epileptic disorder and/or of epileptic seizures. Preferably, the FDC produced in the infusion bag has a volume of about 50-500 ml, preferably about 50-150 ml, more preferably about 100 ml, and provides LEV and LCM in a concentration ready for infusion to a patient within a period of between 10 and 20 minutes, preferably within about 15 minutes.

One aspect of the present invention is a method of preventing, alleviating and/or treating of epileptogenesis, of an epileptic disorder and/or of epileptic seizures, comprising administering to a patient in need thereof an FDC comprising (a) LCM in an amount of 50 mg-400 mg, and preferably in an amount selected from 50 mg, 100 mg, 150 mg and 200 mg and, (b) LEV in an amount of 250-1500 mg, preferably selected from among 250 mg, 500 mg, 750 mg and 1000 mg, wherein said method comprises the parenteral administration of said FDC. In a preferred aspect, the parenteral administration is by injection or by infusion, and particularly preferable by infusion.

In a preferred embodiment, in said method of treatment the injectable or infusible FDC (iFDC) is prepared from a concentrated solution comprising LEV and LCM, with a concentration of LEV of between 2.5 and 10 wt %, and of LCM of between 1 and 5 wt %, preferably of between about 1 and 2 wt %, which requires dilution prior to its administration, preferably by infusion. In just another preferred embodiment the injectable or infusible FDC (iFDC) in said method of treatment is provided by dissolving a dry mixture comprising LEV and LCM, optionally together with pharmaceutically acceptable excipients, in an isotonic buffer prior to administration to a patient.

In an alternative embodiment, in said method of preventing, alleviating and/or treating of epileptogenesis, of an epileptic disorder and/or of epileptic seizures, comprises administering to a patient in need thereof an iFDC which comprises a ready-for-use solution comprising LEV and LCM in a buffer solution having a volume which is ready for infusion, typically between 50 and 500 ml, preferably about 50-150 ml, more preferably about 100 ml. Typical amounts of LCM in the ready-to-use iFDC are 50 mg, 100 mg, 150 mg, 200 mg or 400 mg; typical amounts of LEV are 250 mg, 500 mg, 750 mg, 1000 mg or 1500 mg. Typical buffers are acetate, phosphate, citrate, carbonate, which may or may not include stabilizers, or preservatives such as benzylic acid, phenol, parabens, chlorobutanol, chlorocresol, benzalkonium chloride, thimerosal, phenoxyethanol, if needed.

In another preferred embodiment the injectable or infusible FDC (iFDC) in said method of treatment is provided by mixing (a) a concentrated solution of LEV with a concentration of LEV of between about 2.5 and 10 wt %, and (b) a concentrated solution of LCM with a concentration of LCM of between about 1 and 5 wt %, preferably of between about 1 and 2 wt %, and preferably buffered to a PH of 4.5 to 5.5, with (c) isotonic buffer prior to the administration of said iFDC to the patient, preferably by infusion. In one aspect, the components of the iFDC in said method are mixed together in a multi-chamber infusion bag. Typically the total volume of the final solution is between about 50 ml and 500 ml, preferably between about 50 ml and 150 ml, and is more preferably about 100 ml. In one embodiment, the infusion of the iFDC to the patient will take place in a period of time of between 10 and 20 minutes, preferably in about 15 minutes.

In just another preferred embodiment the injectable or infusible FDC (iFDC) in said method of treatment is provided by mixing (a) a ready-to-use solution of LEV with (b) a ready-to-use solution of LCM, which solutions may optionally be stored in separate chambers of a multi-chamber infusion bag prior to mixing.

### Therapeutic Application

The FDCs as described herein may be used in any condition in which LCM and LEV can be used.

For example, LCM is effective against epileptic conditions, is FDA-approved for epilepsy monotherapy, and is approved in many countries for the adjunctive treatment of partial onset seizures with and without secondary generalization. In addition, LCM has reported efficacy at least in single cases of primary generalized seizures, such as e.g. PGTCS, as well as in acute repetitive seizures, and status epilepticus. LCM may also be useful in the prevention of epileptogenesis following brain insults such as e.g. caused by status epilepticus, see e.g. WO2007/14419 although it is not yet approved in this indication.

LCM has also been reported to show activity in various preclinical models of pain such as cancer pain, chemotherapy-induced pain, neuropathic pain, including particularly diabetic neuropathic pain, trigeminal neuralgia, atypical face pain, migraine, or muscle pain. Moreover, LCM was active in animal models of myotonia, dyskinesia, tinnitus, stroke, arthritis, tremor, and psychoses.

LEV is approved for the treatment of partial onset seizures in patients with epilepsy, as adjunctive therapy for myoclonic seizures in patients with Juvenile Myoclonic Epilepsy (JME), and as adjunctive therapy for the treatment of Primary Generalised Tonic-Clonic (PGTC) seizures in patients with Idiopathic Generalised Epilepsy (IGE).

Hence, one aspect of the present invention is an FDC according to the present disclosure for use in the prevention, alleviation and/or treatment of an epileptic condition, pain, migraine, tinnitus, psychoses, and/or myotonia.

One aspect of the present invention relates to a method of preventing, alleviating or treating a condition selected from epilepsy, epileptic conditions, pain, migraine, tinnitus, psychoses and myotonia by administering to a patient in need thereof a therapeutically effective amount of an FDC comprising LEV and LCM as further described herein.

In one specific aspect the FDC is for use in the prevention, alleviation and/or treatment of epilepsy, of epileptic seizures comprising partial onset seizures with and without secondary generalization, primary generalized epileptic seizures, myoclonic seizures, clonic seizures, tonic seizures, tonic-clonic seizures, atonic seizures, acute repetitive seizures, status epilepticus, and absence seizures.

In a preferred aspect, the FDC is for use in the prevention, alleviation and/or treatment of epilepsy, of epileptic seizures comprising partial onset seizures with and without secondary generalization, of primary generalized epileptic seizures, including in particular myoclonic seizures, clonic seizures, tonic seizures, tonic-clonic seizures, and acute repetitive seizures. In a particularly preferred aspect, the FDC, in particular the oFDC or iFDC, is for the alleviation and/or treatment of partial onset seizures with and without secondary generalization.

In one aspect, the FDC is for use in the treatment, alleviation and/or prevention of epileptogenesis following brain insults, such as e.g. injuries, stroke, intoxication, or status epilepticus.

One aspect of the present invention relates to a method of preventing, alleviating or treating a disease or condition selected from epilepsy, epileptic conditions, pain, migraine, tinnitus, psychoses and/or myotonia by administering to a patient in need thereof a therapeutically effective amount of an FDC comprising LEV and LCM as further described herein. In one aspect said condition or disease is selected from the group of epilepsy, epileptic seizures comprising partial onset seizures with and without secondary generalization, primary generalized epileptic seizures, myoclonic seizures, clonic seizures, tonic seizures, tonic-clonic seizures, atonic seizures, acute repetitive seizures, status epilepticus, and absence seizures.

One aspect of the present invention relates to a method of preventing, alleviating or treating a disease or condition selected from epilepsy, epileptic seizures comprising partial onset seizures with and without secondary generalization, primary generalized epileptic seizures, including in particular myoclonic seizures, clonic seizures, tonic seizures, tonic-clonic seizures, and acute repetitive seizures a therapeutically effective amount of an FDC comprising LEV and LCM as further described herein. In one aspect, said method comprises the treatment, alleviation and/or prevention of epileptogenesis following brain insults, such as e.g. injuries, stroke, intoxication, or status epilepticus.

The pharmaceutical compositions (FDCs) of the present invention can be produced according to various methods known to persons skilled in the art.

In a preferred embodiment, if the FDC is a tablet, the manufacturing process is dry granulation. For example, all excipients and the active ingredients may be mixed, sieved, and lubricated, and the resulting mixture compressed and processed to the final dosage form, e.g. tableted. The lubricant may be added to a pre-mixture of the active ingredients and the other excipients (e.g. disintegrant, glider and optionally binder). The compression process may be performed using a roller compactor. Compression forces are typically selected between about 1000 and 3000 DaN such tablets with a hardness of around 100N are produced.

The invention is further illustrated by the following, non-limiting examples.

### EXAMPLES

### Comparative Example 1

The composition of the commercially available Vimpat® tablets are shown in Table 7. As can be seen from these examples, the drug load of the presently commercially available LCM tablets is only about 40 wt %, while the amount of excipients in the core is more than 50 wt %.

TABLE 7

| Components | 50 mg | 100 mg | 150 mg | 200 mg |
|---|---|---|---|---|
| (all amounts in | Vimpat ® | Vimpat ® | Vimpat ® | Vimpat ® |
| mg/tablet) | tablet | tablet | tablet | tablet |
| Lacosamide | 50.00 | 100.00 | 150.00 | 200.00 |
| Cellulose, | 45.30 | 90.60 | 135.90 | 181.20 |
| microcrystalline | | | | |
| Hydroxypropylcellulose | 13.50 | 27.00 | 40.50 | 54.00 |
| Crospovidone | 10.00 | 20.00 | 30.00 | 40.00 |
| Magnesium stearate | 1.20 | 2.40 | 3.60 | 4.80 |
| Total (tablet core) | 120.00 | 240.00 | 360.00 | 480.00 |

Coating:

| | | | | |
|---|---|---|---|---|
| Total (coating) | 6.00 | 12.00 | 18.00 | 24.00 |
| Total (film-coated tablet) | 126.00 | 252.00 | 378.00 | 504.00 |

### Examples 1-14

### Exemplary oFDCs

Exemplary compositions of oFDCs according to the present invention are given in Examples 1-14, see Tables 8a and 8b.

All oFDCs were produced according to the method described in Example 16. The in vitro dissolution rates of LEV and LCM were measured at the indicated times according to the method described in Example 15. All tablets fulfilled the requirement of a hardness of at least 80 N.

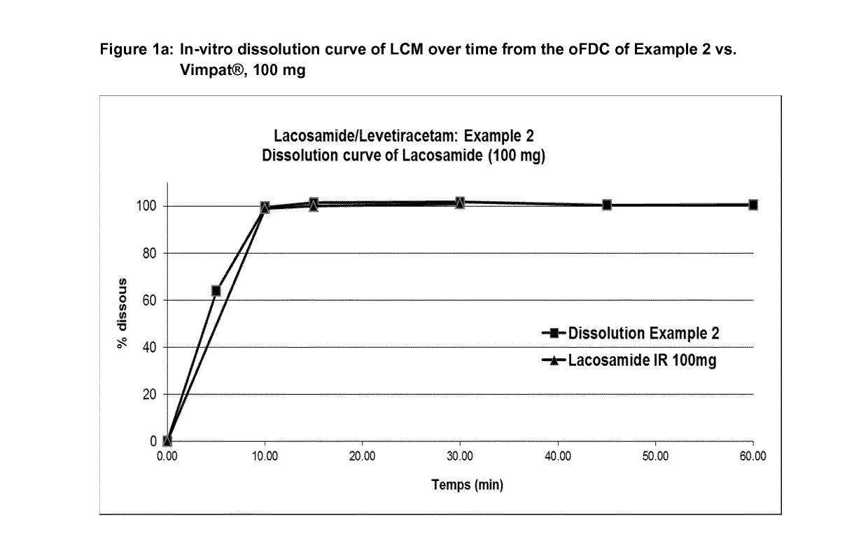

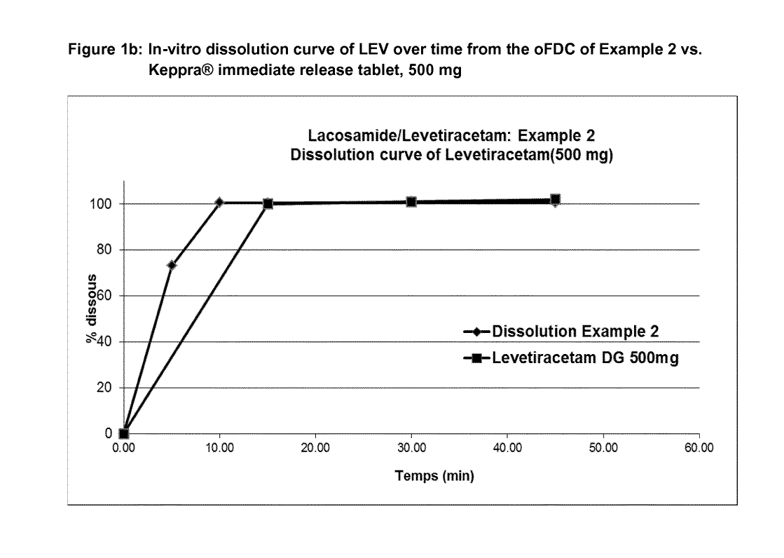

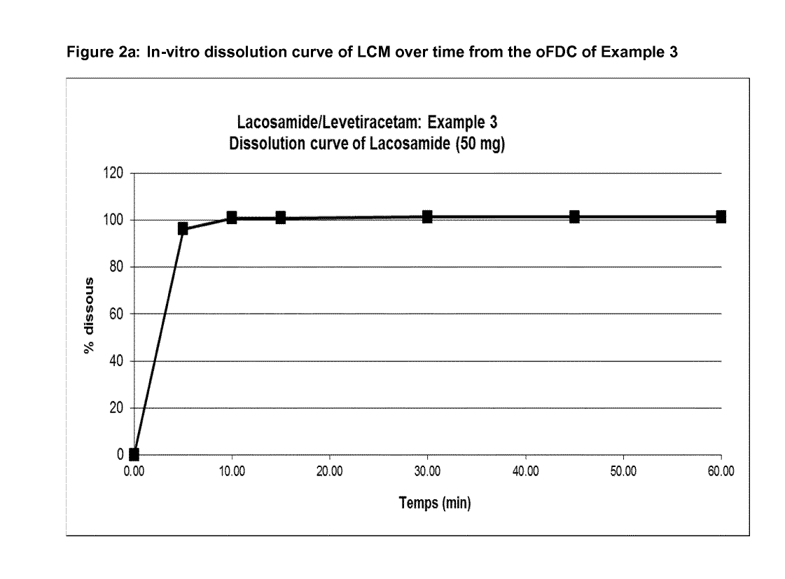

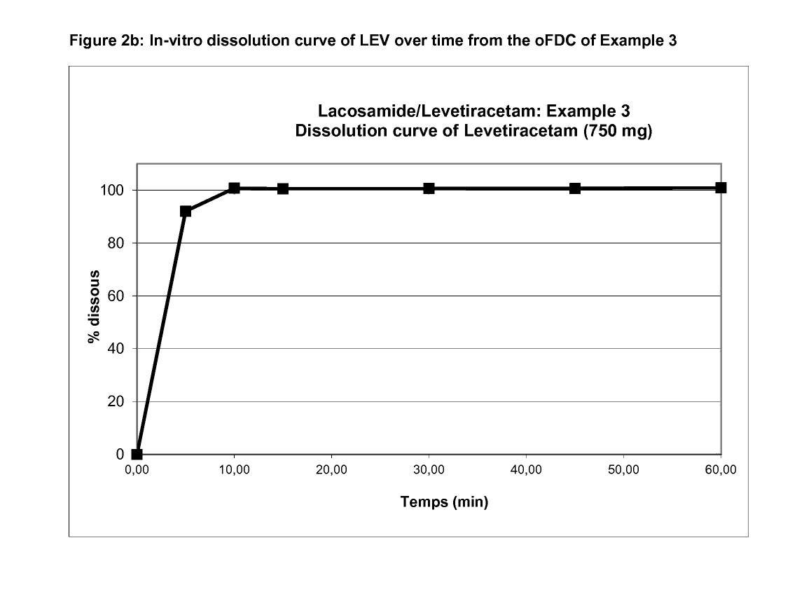

Examples of dissolution profiles are given in FIGS. 1-2.

TABLE 8a

| | Ex-1 | Ex-2 | Ex-3 | Ex-4 | Ex-5 | Ex 6 | Ex-7 | Ex-8 | Ex-14 |
|---|---|---|---|---|---|---|---|---|---|
| LEV (mg) | 500 | 500 | 750 | 750 | 1000 | 250 | 625 | 250 | 1000 |
| LCM (mg) | 50 | 100 | 50 | 100 | 200 | 50 | 125 | 200 | 50 |
| % Colloidal silica 200 | 1 | 1 | 1 | 1 | 1 | 1 | 1 | 1 | 1 |
| % Crospovidone | 2.5 | 2.5 | 2.5 | 2.5 | 5.25 | 5.25 | 5.25 | 5.25 | 5.25 |
| % Sodium Stearyl Fumarate | 1.20 | 1.20 | 1.20 | 1.20 | 1.20 | 1.20 | 1.20 | 1.20 | 1.20 |
| % Dissolution rate LEV after 15′ (min) | 101 | 101.1 | 100.0 | 100.7 | 101 | 102 | 100 | 102 | 98.8 |
| % Dissolution rate LCM after 15′ | 98.7 | 102.5 | 106.6 | 107.9 | 103 | 102 | 105 | 99 | 106 |
| Friability Rate (%)^{a} | 0.21 | 0.21 | 0.46 | 0.34 | 0.12 | 0.08 | 0.15 | 0.13 | 0.6 |

TABLE 8b

| | Function | Ex-9 | Ex-10 | Ex-11 | Ex-12 | Ex-13 |
|---|---|---|---|---|---|---|
| LEV (mg) | API | 250 | 250 | 250 | 250 | 250 |
| LCM (mg) | API | 200 | 200 | 200 | 200 | 200 |
| % PEG 600 | Binder | — | — | 1% | — | — |
| % Colloidal silica 200 | Glidant | 0.76 | 0.76 | 0.76 | 1 | 1 |
| % Croscarmellose | Disintegrating | — | 2.6 | 2.1 | | 8 |
| % Crospovidone | Disintegrating | 2.6 | — | — | 8 | — |
| % Magnesium stearate | Lubricant | — | — | 1.4 | — | — |
| % Sodium Stearyl Fumarate (SSF) | Lubricant | 1.9 | 1.9 | — | 1 | 1 |
| % Dissolution rate LEV after 15′ | | 103 | 97 | 94 | 100 | 111 |
| % Dissolution rate LCM after 15′ | | 95 | 90 | 89 | 90 | 102 |
| Friability Rate^{a,} | | 0.06 | 0.21 | 0.22 | 0.13 | 0.15 |

^{a}Tablet friability is calculated as the percentage of loss of tablets after 100 rotations (at 4 minutes).
A maximum friability of no more than 1% is considered acceptable.

### Example 15

### LEV as Disintegrant to LCM

Table 9 illustrates the surprising effect of LEV on the disintegration time of the tablets. Examples 5, 6, 7 and 14 show a significantly faster disintegration compared to Example 8 which has the same composition but the lower content of LEV.

TABLE 9

| | Ex-5 | Ex-14 | Ex-7 | Ex-8 | Ex-6 |
|---|---|---|---|---|---|
| LEV (mg) | 1000 | 1000 | 625 | 250 | 250 |
| LCM (mg) | 200 | 50 | 125 | 200 | 50 |
| % Colloidal silica 200 | 1.00 | 1.00 | 1.00 | 1.00 | 1.00 |
| % Crospovidone | 5.25 | 5.25 | 5.25 | 5.25 | 5.25 |
| % Sodium Stearyl | 1.20 | 1.20 | 1.20 | 1.20 | 1.20 |
| Fumarate (SSF) | | | | | |
| Tablet disintegration time | 0.48 | 1.22 | 0.77 | 3.68 | 0.85 |
| (min)^{a)b)} | | | | | |

^{a)}Examples were measured following the compression of the oFDCs with a compression force of 2500 daN.
^{b)}Tablet disintegration time is calculated as an average of 6 individuals.

### Example 16

### Description of In-Vitro Dissolution Assay

Dissolutions were measured by dissolving the oFDC in 900 ml of 50 mM potassium dihydrogenate phosphate buffer pH 6.8, in USP Type II apparatus (paddle) at 50 RPM with Japanese sinkers, and by measuring the drug release via HPLC.

### Example 17

### Description of Manufacturing Process

At first, the anhydrous colloidal silica and the disintegrating agent (e.g. croscarmellose or crospovidone) were mixed together and passed through a sieve of 1.0 mm. Then the mixture was added to LEV and LCM in a planetary mixer [Collette MP45] and mixed during 20 minutes at speed 1. The lubricant was then added to the blend and mixed for 3 minutes at speed 1. The blend was transferred to a roller compactor [Minipactor Gerteis] and compacted at 7 kN/cm. The resulting ribbons were passed through a sieve of 1.0 mm and were loaded into a power assisted rotary tabletting press [Courtoy R090] using a tabletting force of about 1000-3000 daN.

### Examples 18a and 18b

### Example iFDC

Fixed dose combinations prepared for administration by infusion or by injection may be prepared by conventional techniques using standard excipients suited for parenteral administration which are well known by persons skilled in the art.

Examples of iFDCs which are either ready for use through a multichamber bag, or which are based on API powders or solids that need to be dissolved in physiological, isotonic buffer prior to administration, respectively, are given in tables 10 and 11 below:

TABLE 10
Fixed dose combination (ready to use
solutions in different chambers)

| Example 18a | Function | 100 mL bag (mg) |
|---|---|---|
| Chamber 1: | | |
| LEV mg | API | 500 |
| Sodium Chloride | Tonicity agent | 600 |
| Citrate and Phosphate buffer | pH adjustment | Ad pH 6.0 |
| WFI (water for injection) | Solvent | To 100 mL |
| Chamber 2 | | |
| LCM | API | 200 |
| Sodium Chloride | Tonicity agent | 600 |
| Sodium Acetate•3H₂O | pH adjustment | 164 |
| Acetic acid glacial | pH adjustment | Ad pH 5.0 |
| WFI (water for injection) | Solvent | To 100 mL |

The content of both chambers will be mixed immediately before or during administration to the patient.

### Example 18b

LCM and LEV drug substance will be mixed as dry substances in suitable ratios as described herein (including but not limited to the LCM-LEV ratios in table 6 above), optionally together with at least one suitable excipient. The solid, essentially water-free mixture is filled in vial. Prior to administration, the drug mixture is dissolved in 100 ml of a suitable isotonic buffer, or water for injection. An example of a composition ready for administration to a patient is given in Table 11:

TABLE 11
Fixed dose combination ready for injection or infusion

| | | |
|---|---|---|
| LEV mg | API | 500 |
| LCM mg | API | 200 |
| Sodium Chloride | Tonicity agent | 600 |
| Sodium Acetate•3H₂O | pH adjustment | 164 |
| Acetic acid glacial | pH adjustment | Ad pH 5.0 to 6.0 |
| WFI (water for injection) | Solvent | To 100 mL |

### Example 19

### Compatibility Studies

Binary mixtures of LCM and LEV (1:1 wt/wt) were stored under several stress conditions in order to evaluate their compatibility. The compatibility study was performed during 6 weeks at 40° C./75% RH in opened containers and at 70° C. dry in closed containers, and probes were taken at T0, and after one, three and six weeks storage, and the probe was inspected optically, as well as for drug content, known impurities and potential impurities using HPLC and LC/MS methods, respectively.

Result: The appearance of the white powder of the 1:1 LEV LCM mixture was unaffected by the storage for 6 weeks under both storage conditions. Also, no impurities could be detected after one, three, and six weeks of storage at 40° C./75% RH in opened containers, and at 70° C. dry in closed containers.

Accordingly, the mixture of LEV-LCM was stable even under stress conditions.

### Example 20

### FDC Stability Studies

Five representative Examples (5-8 and 14; uncoated) as have been put under accelerated conditions (40° C./75% relative humidity) in open dishes for 4 weeks.

The following analyses were performed:
Appearance of tablets
Dissolution test
Chemical degradation products of lacosamide and levetiracetam

### Results:

Appearance There was no change in the appearance of the tablets after 4 weeks at 40° C./75% RH.

### Dissolution

Dissolution results which have been obtained in dissolution assays immediately after production (“T0”) of the FDCs, and after 4 weeks (“T4w”) of storage of the FDCs at 40° C./75% RH in open dishes are presented in tables 12a and 12b. There was no change in the dissolution profile for both, Lacosamide and Levetiracetam, after 4 weeks storage at 40° C./75% RH except for Example 8 (LEV/LCM=250 mg/200 mg) for which the dissolution profile of both compounds was slower compared to T0 during the first 30 minutes.

TABLE 12a

| Examples after | Lacosamide dissolution results | | | | | | |
|---|---|---|---|---|---|---|---|
| production (T0), and | (% of LCM release after indicated period of time) | | | | | | |
| after 4 weeks of | | 5 | 10 | 15 | 30 | 45 | 60 |
| storage (T4 w) | 0 | min | min | min | min | min | min |
| Example 5 T0 | 0.0 | 82.6 | 102.0 | 103.0 | 103.6 | 103.5 | 103.3 |
| Example 5 - T4 w | 0.0 | 92.7 | 104.1 | 105.8 | 106.7 | 106.6 | 106.7 |
| Example 14- T0 | 0.0 | 84.9 | 99.3 | 100.0 | 99.9 | 100.1 | 100.1 |
| Example 14-T4 w | 0.0 | 100.9 | 105.1 | 106.6 | 106.7 | 106.2 | 106.9 |
| Example 7-T0 | 0.0 | 94.4 | 104.4 | 104.6 | 104.8 | 104.8 | 104.7 |
| Example 7-T4 w | 0.0 | 98.1 | 107.8 | 108.4 | 108.6 | 107.6 | 107.6 |
| Example 8-T0 | 0.0 | 51.2 | 90.4 | 99.3 | 101.3 | 101.7 | 101.7 |
| Example 8-T4 w | 0.0 | 18.4 | 38.4 | 54.0 | 81.0 | 93.6 | 98.8 |
| Example 6-T0 | 0.0 | 92.0 | 101.7 | 102.2 | 102.2 | 102.1 | 102.0 |
| Example 6-T4 w | 0.0 | 96.3 | 105.1 | 105.6 | 105.8 | 105.8 | 105.9 |

TABLE 12b

| Examples after | Levetiracetam dissolution results | | | | | | |
|---|---|---|---|---|---|---|---|
| production (T0), and | (% of LEV release after indicated period of time) | | | | | | |
| after 4 weeks of | | 5 | 10 | 15 | 30 | 45 | 60 |
| storage (T4 w) | 0 | min | min | min | min | min | min |
| Example 5 T0 | 0.0 | 86.5 | 100.8 | 101.4 | 101.7 | 101.3 | 101.5 |
| Example 5 - T4 w | 0.0 | 93.7 | 100.6 | 100.8 | 101.3 | 100.9 | 101.2 |
| Example 14- T0 | 0.0 | 88.8 | 101.1 | 101.5 | 101.3 | 101.7 | 101.5 |
| Example 14-T4 w | 0.0 | 96.6 | 96.3 | 98.8 | 98.8 | 98.8 | 98.9 |
| Example 7-T0 | 0.0 | 94.1 | 100.9 | 101.2 | 101.2 | 101.0 | 101.4 |
| Example 7-T4 w | 0.0 | 95.7 | 102.5 | 100.8 | 100.9 | 101.8 | 101.5 |
| Example 8-T0 | 0.0 | 57.3 | 95.7 | 101.8 | 102.6 | 102.7 | 102.2 |
| Example 8-T4 w | 0.0 | 26.4 | 46.8 | 62.0 | 86.9 | 96.5 | 100.4 |
| Example 6-T0 | 0.0 | 96.2 | 102.1 | 102.2 | 102.3 | 102.3 | 102.3 |
| Example 6-T4 w | 0.0 | 98.0 | 101.4 | 101.4 | 101.5 | 101.4 | 101.8 |

### Chemical Stability of Levetiracetam and Lacosamide:

Chemical impurities were examined by HPLC. No impurities were detected in any of the five examined formulations after 4 weeks storage at 40° C./75% RH in open dishes.

### Examples 21-26

### Modified Release oFDCs

Several oFDCs have been produced which release LEV and LCM in a delayed fashion as compared to the IR formulations of Vimpat® and Keppra®, respectively. Exemplary compositions are shown in Table 13 below:

| | Example 21 | | Example 22 | | Example 23 | | Example 24 | | Example 25 | | Example 26 | |
|---|---|---|---|---|---|---|---|---|---|---|---|---|
| | mg | wt % | mg | wt % | mg | wt % | mg | wt % | mg | wt % | mg | wt % |
| LEV | 500 | 50.3 | 500 | 50.3 | 500 | 77.5 | 500 | 59.36 | 500 | 77.5 | 500 | 59.36 |
| LCM | 50 | 5 | 50 | 5.0 | 50 | 7.8 | 50 | 5.94 | 50 | 7.8 | 50 | 5.94 |
| Aerosil 200 | 9.95 | 1 | 9.95 | 1.0 | 6.4 | 1.0 | 8.42 | 1.00 | 6.45 | 1.0 | 8.42 | 1.00 |
| Crospovidon | 24.86 | 2.5 | 24.86 | 2.5 | 16.1 | 2.5 | 21.06 | 2.50 | 16.12 | 2.5 | 21.06 | 2.50 |
| Sodium | 11.93 | 1.2 | 11.93 | 1.2 | 7.7 | 1.2 | 10.11 | 1.20 | 7.74 | 1.2 | 10.11 | 1.20 |
| stearyl | | | | | | | | | | | | |
| fumarate | | | | | | | | | | | | |
| Methocel | 298.37 | 30 | 99.46 | 10 | 0.0 | 0.0 | 0.00 | 0.00 | 64.48 | 10.0 | 252.68 | 30.00 |
| K100 LV CR | | | | | | | | | | | | |
| Methocel | 99.46 | 10 | 298.4 | 30 | 64.5 | 10.0 | 252.7 | 30.00 | 0.00 | 0.0 | 0.00 | 0.00 |
| K15M CR | | | | | | | | | | | | |
| Calculated | 994.6 | 100 | 994.6 | 100 | 644.8 | 100 | 842.3 | 100 | 644.8 | 100 | 842.27 | 100.00 |
| tablet mass | | | | | | | | | | | | |
| (mg) | | | | | | | | | | | | |

Examples 21-26 were prepared by mixing all components and by roller compacting and tableting the blend to obtain the oFDCs in the form of tablets, see Example 17.

The release profiles of LEV and LCM, respectively, were measured in the in vitro dissolution assay described in Example 16 herein. The release profiles of the oFDCs of Examples 21-26 are shown in FIGS. 3a and 3b, and Table 14.

TABLE 14

| | % Release of LEV/LCM at the indicated point in time | | | | | |
|---|---|---|---|---|---|---|
| | 1 | 2 | 4 | 6 | 8 | 12 |
| | hr | hr | hr | hr | hr | hr |
| Ex 21 | 31/19 | 47/31 | 67/48 | 82/61 | 91/72 | 100/87 |
| Ex22 | 29/19 | 44/30 | 62/38 | 75/58 | 84/68 | 94/92 |
| Ex23 | 43/31 | 65/49 | 89/74 | 98/89 | 100/95 | 100/96 |
| Ex24 | 33/20 | 48/32 | 69/50 | 82/63 | 91/74 | 99/88 |
| Ex25 | 84/81 | 100/100 | 100/100 | 100/100 | 100/100 | 100/100 |
| Ex26 | 37/26 | 56/42 | 80/64 | 93/79 | 99/89 | 100/96 |

As one can see, all MR oFDCs show a delayed release of LEV and LCM compared to the commercial Keppra and Vimpat IR formulations. Preferred are those oFDCs which show dissolution profiles of LEV and LCM which match the specification given in table 15 below.

TABLE 15
Target dissolution profile of LCM
and LEV modified release oFDCs

| Dissolution time | 1 h | 2 h | 4 h | 8 h | 12 h |
|---|---|---|---|---|---|
| Target Dissolution | 22-45 | 39-60 | 62-82 | 80-100 | 80-100 |
| rate LEV (%) | | | | | |
| Target Dissolution | 9.5-35 | 18-45 | 33-70 | 55-91 | 75-98 |
| rate LCM (%) | | | | | |

### Examples 27 and 28

### Functionally Coated oFDCs

An example of a functionally coated oFDC is given in Tab 16 below:

TABLE 16
Functionally coated oFDC

| Core | mg | wt % |
|---|---|---|
| LEV | 500 | 83.3 |
| LCM | 50 | 8.3 |
| Colloidal silica 200 | 6 | 1 |
| % Crospovidone | 15 | 2.5 |
| % Sodium Stearyl Fumarate | 7.2 | 1.2 |
| Total weight core | 578.2 | 96.3 |

Film coating (Example 27):

| | | |
|---|---|---|
| Eudragit ® NE 40 D1¹ | 11 | 1.8 |
| Talc | 10.1 | 1.7 |
| Colloidal anhydrous silica | 0.6 | 0.1 |
| Total weight coating | 22.8 | 3.6 |
| Total weight oFDC | 600 | 99.99 |

Eudragit¹ NE 40 D (Evonik Röhm GmbH) is an aqueous dispersion with a solid content of 40.0 wt % consisting of neutral ethyl acrylate/metyl methacrylate copolymer (2:1) (38.0 wt-%) and nonoxynol 100 (2.0 wt-%).

Example 28 is based on Example 27 but has a higher amount (3.5 wt %) of Eudragit NE 40D in the film coating.

The core tablets can be produced as described in Example 16. The coating dispersion is prepared by dispersing talc in purified water. Eudragit® NE40D and colloidal anhydrous silica is added and mixed until a homogenous dispersion was obtained. The tablets can be coated in a pan coating system with the coating suspension until the target weight is reached.

The invention is further described by the following items:
1. Pharmaceutical composition for the combined administration of lacosamide and levetiracetam, said composition comprising lacosamide in an amount of 50 mg-400 mg and levetiracetam in an amount of 250-1500 mg, wherein said composition is selected from
   (a) a fixed dose combination for the oral administration of lacosamide and levetiracetam, comprising at least about 80 wt %, preferably at least 85 wt %, more preferably at least 90 wt %, more preferably at least about 93 wt %, even more preferably at least about 95 wt % of active ingredient consisting of levetiracetam and lacosamide in a ratio (wt/wt) of about 1:1 to 20:1, and
   (b) a fixed dose combination for the simultaneous injection or infusion of lacosamide and levetiracetam in a ratio (wt/wt) of about 1:1 to 20:1.
2. Pharmaceutical composition according to item 1, wherein the amount of lacosamide in the formulation is 50 mg, 100 mg, 150 mg and 200 mg, and wherein the amount of levetiracetam is selected from the group of 250, 500, 750 and 1000 mg, and wherein the ratio (wt/wt) of levetiracetam to lacosamide is from about 3:1 to 15:1.
3. Pharmaceutical composition according to any one of the preceding items, said composition being a solid oral fixed dose combination comprising at least about 90 wt % of active ingredient consisting of levetiracetam and lacosamide, relative to the total weight of the composition.
4. Pharmaceutical composition according to any one of the preceding items, said composition being a solid oral fixed dose combination, wherein levetiracetam and lacosamide are contained in the same layer/matrix.
5. Pharmaceutical composition according to any one of the preceding items, said composition being a solid oral fixed dose combination wherein the composition comprises lacosamide and levetiracetam in a total amount of at least 90 wt %, and excipients in a total amount of up to 10 wt %, preferably up to 7 wt %, more preferably up to 5 wt %, wherein the excipients comprise at least one gildant, at least one disintegrant, at least one lubricant, and optionally a binder or diluent.
6. Pharmaceutical composition according to any one of the preceding items, said composition being a solid oral fixed dose combination wherein the composition comprises up to about 7 wt %, preferably up to 5 wt % excipients, consisting of at least one gildant, at least one disintegrant, and at least one lubricant.
7. Pharmaceutical composition according to any one of items 5-6, wherein the glidant is selected from magnesium silicate, magnesium trisilicate, sodium stearate, hydrophobic colloidal silica, magnesium oxide, talc, and colloidal silicon dioxide, preferably in an amount of 0.5-2 wt %.
8. Pharmaceutical composition according to any one of items 5-7, wherein the disintegrant is selected from croscarmellose, crospovidone, sodium starch glycolate, pregelatinized starch, native starch, preferably in an amount of 2-8 wt %.
9. Pharmaceutical composition according to any one of items 7-8, wherein the lubricant is a hydrophilic lubricant selected from sodium stearyl fumarate, sodium laurylsulfate, potassium benzoate, and polyethylene glycol, preferably in an amount of 0.75-2 wt %.
10. Pharmaceutical composition according to any one of the preceding items, wherein the composition is a tablet.
11. Pharmaceutical composition according to any one of the preceding items, said composition being a solid oral fixed dose combination wherein the total volume of the FDC is less than the combined total volume Xn+Yn of the commercial Vimpat® and Keppra® formulations containing the same amounts of lacosamide and levetiracetam, which volumes are given in the following table:

| Commercial Formulation | Drug Load/Dosage | Volume (mm³) |
|---|---|---|
| Vimpat ® | 50 mg | X1 = 99.80 |
| | 100 mg | X2 = 199.59 |
| | 150 mg | X3 = 299.39 |
| | 200 mg | X4 = 399.18 |
| Keppra ® | 250 mg | Y1 = 234.50 |
| | 500 mg | Y2 = 467.19 |
| | 750 mg | Y3 = 701.24 |
| | 1000 mg | Y4 = 949.73 |

12. Pharmaceutical composition according to any one of the preceding items, said composition being a solid oral fixed dose combination and wherein the composition comprises
   (a) at least 93% active ingredient consisting of levetiracetam and lacosamide in a ratio (wt/wt) of about 5:1 to about 15:1
   (b) up to 7 wt % of excipients, said excipients comprising
      (b1) 0.75-2 wt % colloidal silicon dioxide
      (b2) 2-5 wt % crospovidone, and
      (b3) 0.75-2.5 wt of a hydrophilic lubricant, preferably sodium stearyl fumarate.
13. Pharmaceutical composition according to any one of the preceding items, said composition being a solid oral fixed dose combination and wherein the composition provides an in-vitro release of each of lacosamide and levetiracetam, in an amount of at least 85% within 15 minutes, when the in-vitro release of lacosamide and levetiracetam is measured in USP type II apparatus (paddle) using Japanese sinkers, in 900 mL of Phosphate buffer, PH 6.8, at 50 rpm.
14. Pharmaceutical composition according to any one of items 1 or 2, said composition being an injectable or infusible fixed dose combination which comprises (a) lacosamide and levetiracetam in an amount selected from 50 mg, 100 mg, 150 mg and 200 mg, and (b) LEV in an amount selected from 250 mg, 500 mg, 750 mg and 1000 mg, and wherein both drugs are dissolved either (i) in a volume of between 5 and 20 ml which is further diluted to the final injection or infusion volume of between 50 and 500 ml within 48 hours before use, or (ii) in a volume of between about 50 to 500 ml which is ready for infusion to a patient.
15. Kit comprising an infusion bag and the pharmaceutical composition of item 14 which is contained is said bag.
16. Pharmaceutical composition according to one of the previous items, wherein the composition is for use in the prevention, alleviation and/or treatment of epileptogenesis, of an epileptic disorder and/or of epileptic seizures, said epileptic seizures comprising partial onset seizures with and without secondary generalization, primary generalized epileptic seizures, myoclonic seizures, clonic seizures, tonic seizures, tonic-clonic seizures, atonic seizures, and acute repetitive seizures.
17. Method of manufacturing the oral fixed dose combination according to anyone of items 1 to 13, comprising dry mixing and compacting the ingredients.
18. Oral fixed dose combination comprising levetiracetam and lacosamide, for use as a medicine which use comprises the twice daily administration of
   (i) one entity per administration of said fixed dosage combination, wherein said one entity provides the combined release of lacosamide and levetiracetam in dosages selected from (a) 50 mg LCM+250 mg LEV, (b) 50 mg LCM+500 mg LEV, (c) 50 mg LCM+750 mg LEV, (d) 100 mg LCM+500 mg LEV, (e) 100 mg LCM+750 mg LEV, and (f) 200 mg LCM+1000 mg LEV, (g) 150 mg LCM+500 mg LEV, (h) 150 mg LCM+1000 mg LEV, and (i) 200 mg LCM+500 mg LEV, (j) 100 mg LCM+250 mg LEV, (k) 100 mg LCM+1000 mg LEV, (l) 150 mg LCM+750 mg LEV, and (m) 200 mg LCM+750 mg LEV, or
   (ii) two entities per administration of the fixed dosage combination, each entity of which provides the combined release of lacosamide and levetiracetam in a dosage selected from (a) 50 mg LCM+750 mg LEV, (b) 100 mg LCM+750 mg LEV, 50 mg LCM and 500 mg LEV, or 50 mg LCM and 750 mg LEV.
19. Oral fixed dose combination according to item 18 which is a tablet.
20. Oral fixed dose combination according to any one of items 18-19, wherein the oral fixed dose combination provides an in-vitro release of each of lacosamide and levetiracetam, in an amount of at least 85% within 15 minutes, when the in-vitro release of lacosamide and levetiracetam is measured in USP type II apparatus (padlde) using Japanese sinkers, in 900 ml of Phosphate buffer, PH 6.8, at 50 rpm.
21. Oral fixed dose combination according to any one of items 18-20, wherein the total amount of lacosamide and levetiracetam in said oral fixed dose combination is at least 90 wt %.
22. Pharmaceutical composition according to items 1-14 comprising multiple unit dosage forms for oral administration, in particular pellets, granules or mini-tablets.
23. Pharmaceutical composition according to anyone of items 1 to 14 wherein the pharmaceutical composition comprises multiple unit dosage forms selected from minitablets, pellets or granules, wherein the drug load of each unit is 50 mg or less, preferably 25 mg, or less.
24. Pharmaceutical composition according to item 23 for use in the oral treatment of partial onset seizures, with and without secondary generalization, or for the treatment of primary generalized tonic-clonic seizures.

1. A pharmaceutical composition for the combined administration of lacosamide and levetiracetam, said composition comprising lacosamide in an amount of 50 mg-400 mg and levetiracetam in an amount of 250-1500 mg, wherein said composition is selected from
(a) a fixed dose combination for the oral administration of lacosamide and levetiracetam, comprising at least about 80 wt %, of active ingredient consisting of levetiracetam and lacosamide in a ratio (wt/wt) of about 1:1 to 20:1, and
(b) a fixed dose combination for the simultaneous injection or infusion of lacosamide and levetiracetam in a ratio (wt/wt) of about 1:1 to 20:1.

2. The pharmaceutical composition according to claim 1, wherein the amount of lacosamide in the formulation is 50 mg, 100 mg, 150 mg or 200 mg, and wherein the amount of levetiracetam is selected from the group of 250, 500, 750 and 1000 mg, and wherein the ratio (wt/wt) of levetiracetam to lacosamide is from about 3:1 to 15:1.

3. The pharmaceutical composition according to claim 1, wherein said composition is a solid oral fixed dose combination comprising at least about 90 wt % of active ingredient consisting of levetiracetam and lacosamide, relative to the total weight of the composition.

4. The pharmaceutical composition according to claim 1, wherein said composition is a solid oral fixed dose combination, wherein levetiracetam and lacosamide are contained in the same layer/matrix.

5. The pharmaceutical composition according to claim 1, said composition being a solid oral fixed dose combination wherein the composition comprises lacosamide and levetiracetam in a total amount of at least 90 wt %, and excipients in a total amount of up to 10 wt %, wherein the excipients comprise at least one glidant, at least one disintegrant, at least one lubricant, and optionally a binder or diluent.

6. The pharmaceutical composition according to claim 1, said composition being a solid oral fixed dose combination wherein the composition comprises up to about 7 wt % excipients, consisting of at least one glidant, at least one disintegrant, and at least one lubricant.

7. The pharmaceutical composition according to claim 5, wherein the glidant is selected from the group consisting of magnesium silicate, magnesium trisilicate, sodium stearate, hydrophobic colloidal silica, magnesium oxide, talc, and colloidal silicon dioxide, in an amount of 0.5-2 wt %.

8. The pharmaceutical composition according to claim 5, wherein the disintegrant is selected from the group consisting of croscarmellose, crospovidone, sodium starch glycolate, pregelatinized starch, and native starch, in an amount of 2-8 wt %.

9. The pharmaceutical composition according to claim 7, wherein the lubricant is a hydrophilic lubricant selected from the group consisting of sodium stearyl fumarate, sodium laurylsulfate, potassium benzoate, and polyethylene glycol, in an amount of 0.75-2 wt %.

10. The pharmaceutical composition according to claim 1, wherein the composition is a tablet.

11. The pharmaceutical composition according to claim 1, said composition being a solid oral fixed dose combination and wherein the composition comprises
(a) at least 93% active ingredient consisting of levetiracetam and lacosamide in a ratio (wt/wt) of about 5:1 to about 15:1,
(b) up to 7 wt % of excipients, said excipients comprising
(b1) 0.75-2 wt % colloidal silicon dioxide
(b2) 2-5 wt % crospovidone, and
(b3) 0.75-2.5 wt of a hydrophilic lubricant.

12. The pharmaceutical composition according to claim 1, said composition being a solid oral fixed dose combination and wherein the composition provides an in-vitro release of each of lacosamide and levetiracetam, in an amount of at least 85% within 15 minutes, when the in-vitro release of lacosamide and levetiracetam is measured in USP type II apparatus (paddle) using Japanese sinkers, in 900 ml of Phosphate buffer, PH 6.8, at 50 rpm.

13. (canceled)

14. A method of manufacturing the oral fixed dose combination according to claim 1, comprising dry mixing and compacting the ingredients.

15. A dosing regimen for the administration of an oral fixed dose combination comprising levetiracetam and lacosamide, for use in the prevention, alleviation and/or treatment of an epileptic disorder and/or of epileptic seizures wherein the dosing regimen comprises the twice daily administration of
(i) one entity per administration of said fixed dosage combination, wherein said one entity provides the combined release of lacosamide and levetiracetam in dosages selected from
(a) 50 mg LCM+250 mg LEV, (b) 50 mg LCM+500 mg LEV, (c) 50 mg LCM+750 mg LEV, (d) 100 mg LCM+500 mg LEV, (e) 100 mg LCM+750 mg LEV, (f) 200 mg LCM+1000 mg LEV, (g) 150 mg LCM+500 mg LEV, (h) 150 mg LCM+1000 mg LEV, (i) 200 mg LCM+500 mg LEV, (j) 100 mg LCM+250 mg LEV, (k) 100 mg LCM+1000 mg LEV, (l) 150 mg LCM+750 mg LEV, and (m) 200 mg LCM+750 mg LEV,
or
(ii) two entities per administration of the fixed dosage combination, each entity of which provides the combined release of lacosamide and levetiracetam in a dosage selected from (a) 50 mg LCM+750 mg LEV, (b) 100 mg LCM+750 mg LEV, 50 mg LCM and 500 mg LEV, or 50 mg LCM and 750 mg LEV.

16. The dosing regimen of claim 15, wherein the oral fixed dose combination is a tablet.

17. The dosing regimen of claim 15, wherein the total amount of lacosamide and levetiracetam in said oral fixed dose combination is at least 90 wt %.

18. A method for treating epileptogenesis, an epileptic disorder and/or an epileptic seizure, the method comprising administering to a subject in need thereof a therapeutically effective amount of the pharmaceutical composition of claim 1.

19. The method of claim 18, wherein the epileptic seizure comprises partial onset seizures with and without secondary generalization, primary generalized epileptic seizures, myoclonic seizures, clonic seizures, tonic seizures, tonic-clonic seizures, atonic seizures, or acute repetitive seizures.

20. The pharmaceutical composition according to claim 1, wherein the fixed dose combination for the oral administration of lacosamide and levetiracetam comprises at least about 85 wt % of active ingredient consisting of levetiracetam and lacosamide in a ratio (wt/wt) of about 1:1 to 20:1.

21. The pharmaceutical composition according to claim 1, wherein the fixed dose combination for the oral administration of lacosamide and levetiracetam comprises at least about 90 wt % of active ingredient consisting of levetiracetam and lacosamide in a ratio (wt/wt) of about 1:1 to 20:1.

22. The pharmaceutical composition according to claim 1, wherein the fixed dose combination for the oral administration of lacosamide and levetiracetam comprises at least about 93 wt % of active ingredient consisting of levetiracetam and lacosamide in a ratio (wt/wt) of about 1:1 to 20:1.

23. The pharmaceutical composition according to claim 1, wherein the fixed dose combination for the oral administration of lacosamide and levetiracetam comprises at least about 95 wt % of active ingredient consisting of levetiracetam and lacosamide in a ratio (wt/wt) of about 1:1 to 20:1.
